SEQUENCE LISTING (3 sequences)

SEQ ID NO: 1 - protein, 631 aa, Homo sapiens
MPRYGASLRQSCPRSGREQGQDGTAGAPGLLWMGLVLALALALALALALSDSRVLWAPAEAHPLSPQGHPARLHRIVPRLRDVFGWGNLTCPICKGLFTAINLGLKKEPNVARVGSVAIKLCNLLKIAPPAVCQSIVHLFEDDMVEVWRRSVLSPSEACGLLLGSTCGHWDIFSSWNISLPTVPKPPPKPPSPPAPGAPVSRILFLTDLHWDHDYLEGTDPDCADPLCCRRGSGLPPASRPGAGYWGEYSKCDLPLRTLESLLSGLGPAGPFDMVYWTGDIPAHDVWHQTRQDQLRALTTVTALVRKFLGPVPVYPAVGNHESTPVNSFPPPFIEGNHSSRWLYEAMAKAWEPWLPAEALRTLRIGGFYALSPYPGLRLISLNMNFCSRENFWLLINSTDPAGQLQWLVGELQAAEDRGDKVHIIGHIPPGHCLKSWSWNYYRIVARYENTLAAQFFGHTHVDEFEVFYDEETLSRPLAVAFLAPSATTYIGLNPGYRVYQIDGNYSGSSHVVLDHETYILNLTQANIPGAIPHWQLLYRARETYGLPNTLPTAWHNLVYRMRGDMQLFQTFWFLYHKGHPPSEPCGTPCRLATLCAQLSARADSPALCRHLMPDGSLPEAQSLWPRPLFC

SEQ ID NO: 2 - protein, 585 aa, Homo sapiens
LALSDSRVLWAPAEAHPLSPQGHPARLHRIVPRLRDVFGWGNLTCPICKGLFTAINLGLKKEPNVARVGSVAIKLCNLLKIAPPAVCQSIVHLFEDDMVEVWRRSVLSPSEACGLLLGSTCGHWDIFSSWNISLPTVPKPPPKPPSPPAPGAPVSRILFLTDLHWDHDYLEGTDPDCADPLCCRRGSGLPPASRPGAGYWGEYSKCDLPLRTLESLLSGLGPAGPFDMVYWTGDIPAHDVWHQTRQDQLRALTTVTALVRKFLGPVPVYPAVGNHESTPVNSFPPPFIEGNHSSRWLYEAMAKAWEPWLPAEALRTLRIGGFYALSPYPGLRLISLNMNFCSRENFWLLINSTDPAGQLQWLVGELQAAEDRGDKVHIIGHIPPGHCLKSWSWNYYRIVARYENTLAAQFFGHTHVDEFEVFYDEETLSRPLAVAFLAPSATTYIGLNPGYRVYQIDGNYSGSSHVVLDHETYILNLTQANIPGAIPHWQLLYRARETYGLPNTLPTAWHNLVYRMRGDMQLFQTFWFLYHKGHPPSEPCGTPCRLATLCAQLSARADSPALCRHLMPDGSLPEAQSLWPRPLFC

SEQ ID NO: 3 - DNA, 2410 bp, Homo sapiens
agtcagccgactacagagaagggtaatcgggtgtccccggcgccgcccggggccctgagggctggctagggtccaggccgggggggacgggacagacgaaccagccccgtgtaggaagcgcgacaatgccccgctacggagcgtcactccgccagagctgccccaggtccggccgggagcagggacaagacgggaccgccggagcccccggactcctttggatgggcctggtgctggcgctggcgctggcgctggcgctggcgctggctctgtctgactctcgggttctctgggctccggcagaggctcaccctctttctccccaaggccatcctgccaggttacatcgcatagtgccccggctccgagatgtctttgggtgggggaacctcacctgcccaatctgcaaaggtctattcaccgccatcaacctcgggctgaagaaggaacccaatgtggctcgcgtgggctccgtggccatcaagctgtgcaatctgctgaagatagcaccacctgccgtgtgccaatccattgtccacctctttgaggatgacatggtggaggtgtggagacgctcagtgctgagcccatctgaggcctgtggcctgctcctgggctccacctgtgggcactgggacattttctcatcttggaacatctctttgcctactgtgccgaagccgccccccaaaccccctagccccccagccccaggtgcccctgtcagccgcatcctcttcctcactgacctgcactgggatcatgactacctggagggcacggaccctgactgtgcagacccactgtgctgccgccggggttctggcctgccgcccgcatcccggccaggtgccggatactggggcgaatacagcaagtgtgacctgcccctgaggaccctggagagcctgttgagtgggctgggcccagccggcccttttgatatggtgtactggacaggagacatccccgcacatgatgtctggcaccagactcgtcaggaccaactgcgggccctgaccaccgtcacagcacttgtgaggaagttcctggggccagtgccagtgtaccctgctgtgggtaaccatgaaagcacacctgtcaatagcttccctccccccttcattgagggcaaccactcctcccgctggctctatgaagcgatggccaaggcttgggagccctggctgcctgccgaagccctgcgcaccctcagaattggggggttctatgctctttccccataccccggtctccgcctcatctctctcaatatgaatttttgttcccgtgagaacttctggctcttgatcaactccacggatcccgcaggacagctccagtggctggtgggggagcttcaggctgctgaggatcgaggagacaaagtgcatataattggccacattcccccagggcactgtctgaagagctggagctggaattattaccgaattgtagccaggtatgagaacaccctggctgctcagttctttggccacactcatgtggatgaatttgaggtcttctatgatgaagagactctgagccggccgctggctgtagccttcctggcacccagtgcaactacctacatcggccttaatcctggttaccgtgtgtaccaaatagatggaaactactccgggagctctcacgtggtcctggaccatgagacctacatcctgaatctgacccaggcaaacataccgggagccataccgcactggcagcttctctacagggctcgagaaacctatgggctgcccaacacactgcctaccgcctggcacaacctggtatatcgcatgcggggcgacatgcaacttttccagaccttctggtttctctaccataagggccacccaccctcggagccctgtggcacgccctgccgtctggctactctttgtgcccagctctctgcccgtgctgacagccctgctctgtgccgccacctgatgccagatgggagcctcccagaggcccagagcctgtggccaaggccactgttttgctagggccccagggcccacatttgggaaagttcttgatgtaggaaagggtgaaaaagcccaaatgctgctgtggttcaaccaggcaagatcatccggtgaaagaaccagtccctgggccccaaggatgccggggaaacaggaccttctcctttcctggagctggtttagctggatatgggagggggtttggctgcctgtgcccaggagctagactgccttgaggctgctgtcctttcacagccatggagtagaggcctaagttgacactgccctgggcagacaagacaggagctgtcgccccaggcctgtgctgcccagccaggaaccctgtactgctgctgcgacctgatgctgccagtctgttaaaataaagataagagacttggactcca

(19) United States
(12) Patent Application Publication
(10) Pub. No.: US 2023/0226221 A1
(43) Pub. Date: Jul. 20, 2023

(54) USE OF RECOMBINANT HUMAN ACID SPHINGOMYELINASE TO IMPROVE SKELETAL MYOFIBER REPAIR

(71) Applicant: CHILDREN'S NATIONAL MEDICAL CENTER, Washington, DC (US)

(72) Inventors: Jyoti K. Jaiswal, Rockville, MD (US); Aurelia Defour, Marseille (FR); Daniel Bittel, Washington, DC (US); Goutam Chandra, Silver Spring, MD (US); Sreetama Sen Chandra, Silver Spring, MD (US)

(21) Appl. No.: 18/008,422

(22) PCT Filed: Jun. 29, 2021
(86) PCT No.: PCT/US2021/039537

§ 371 Date: Dec. 5, 2022

Related U.S. Application Data
(60) Provisional application No. 63/046,202, filed on Jun. 30, 2020.

(51) Int. Cl. A61K 48/00; C12N 15/86; A61P 21/00; C12N 9/16

(52) U.S. Cl. CPC A61K 48/0058; A61P 21/00; C12N 9/16; C12N 15/86; C12Y 301/04012

(57) ABSTRACT

Compositions and methods for the treatment of muscular dystrophies are provided.

This application claims priority under 35 U.S.C. §119(e) to U.S. Provisional Patent Application No. 63/046,202, filed Jun. 30, 2020. The foregoing application is incorporated by reference herein.

This invention was made with government support under Grant No. 5R01AR055686 awarded by the National Institute of Arthritis and Musculoskeletal and Skin Diseases (NIAMS). The government has certain rights in the invention.

### FIELD OF THE INVENTION

The present invention relates to myofiber repair, particularly compositions and methods for treating, inhibiting, and/or preventing a dysferlinopathy are provided.

### BACKGROUND OF THE INVENTION

Dysferlinopathy is a progressive muscle wasting disease, such as limb-girdle muscular dystrophy type 2B (LGMD2B) or Miyoshi muscular dystrophy 1, based on its muscle involvement (Bashir, et al. (1998) Nat. Genet., 20:37-42; Liu, et al. (1998) Nat. Genet., 20:31-36). Dysferlin deficit leads to altered vesicle formation and trafficking, poor repair of injured cell membranes, and increased muscle inflammation (Cenacchi, et al. (2005) J. Clin. Pathol., 58:190-195; Demonbreun, et al. (2011) Hum. Mol. Genet., 20:779-789; Bansal, et al. (2003) Nature 423:168-172; Ho, ET AL. (2004) Hum. Mol. Genet., 13:1999-2010; Gallardo, et al. (2001) Neurology 57:2136-2138; Bonnemann, et al. (1996) Curr. Opin. Pediatr., 8:569-582).

Disease severity in dysferlinopathies correlates with fatty replacement of muscle. Indeed, accumulation of fat correlates directly with pathogenesis in dysferlin deficient patients, a property unique to dysferlinopathies. Fibro/adipogenic precursors (FAPs) accumulation also correlates with the disease severity as FAPs cause adipogenic loss of dysferlinopathic muscle (Hogarth, et al. (2019) Nat. Commun., 10:2430). Notably, a deficit in annexin A2 prevents adipogenic loss of dysferlinopathic muscle (Defour, et al. (2017) Hum. Mol. Genet., 26(11):1979-1991). Extracellular annexin A2, by interacting with macrophages, facilitates the adipogenic conversion of dysferlinopathic muscle. Reduced FAP activation may be the basis for reduced adipogenesis in annexin A2 deficient dysferlinopathic muscle. Indeed, blocking FAP adipogenesis restricts adipogenic loss of dysferlinopathic muscle.

Dysferlin contains C2 domains that are found in Ca²⁺-dependent membrane fusion proteins such as synaptotagmins (Lek, et al. (2012) Traffic 13:185-194). Thus, dysferlin may regulate muscle function by regulating vesicle trafficking and fusion (Posey, et al. (2011) Curr. Top. Dev. Biol. 96:203-230; Lennon, et al. (2003) J. Biol. Chem., 278:50466-50473; Kesari, et al. (2008) Am. J. Pathol., 173:1476-1487; Nagaraju, et al. (2008) Am. J. Pathol., 172:774-785). Dysferlin deficiency has also been implicated in conflicting reports regarding the fusion ability of dysferlinopathic myoblasts (Demonbreun, et al. (2011) Hum. Mol. Genet., 20:779-789; de Luna, et al. (2006) J. Biol. Chem., 281:17092-17098; Humphrey, et al. (2012) Exp. Cell. Res., 318:127-135; Philippi, et al. (2012) PLoS Curr., 4:RRN1298).

With such diverse roles for dysferlin, the mechanism through which dysferlin deficiency results in muscle pathology is unresolved. As skeletal muscle-specific re-expression of dysferlin rescues all dysferlinopathic pathologies, myofiber repair has been suggested to be the unifying deficit underlying muscle pathology in dysferlinopathy (Millay, et al. (2009) Am. J. Pathol., 175: 1817-1823; Lostal, et al. (2010) Hum. Mol. Genet., 19:1897-1907; Han, R. (2011) Skelet. Muscle 1:10). Repair of injured cell membranes requires subcellular compartments, which in mammalian cells include lysosomes, enlargeosomes, caveolae, dysferlin-containing vesicles, and mitochondria (Lennon, et al. (2003) J. Biol. Chem., 278:50466-50473; Bansal, et al. (2003) Nature 423:168-172; Borgonovo, et al. (2002) Nat. Cell. Biol., 4:955-962; Corrotte, et al. (2013) Elife 2:e00926; Sharma, et al. (2012) J. Biol. Chem. 287:30455-30467).

Cells from muscular dystrophy patients that have normal dysferlin expression exhibit normal lysosome and enlargeosome exocytosis (Jaiswal, et al. (2007) Traffic 8:77-88). However, dysferlinopathic muscle cells exhibit enlarged LAMP2-positive lysosomes, reduced fusion of early endosomes, altered expression of proteins regulating late endosome/lysosome fusion, and reduced injury-triggered cell-surface levels of LAMP1 (Demonbreun, et al. (2011) Hum. Mol. Genet., 20:779-789; Lennon, et al. (2003) J. Biol. Chem., 278:50466-50473; Kesari, et al. (2008) Am. J. Pathol., 173: 1476-1487). In non-muscle cells, lack of dysferlin reduces lysosomal exocytosis (Han, et al. (2012) J. Cell. Sci., 125: 1225-1234). These findings implicate lysosomes in dysferlin-mediated muscle cell membrane repair (Corrotte, et al. (2013) Elife 2:e00926; McDade, et al. (2013) Hum. Mol. Genet., 23:1677-1686).

Despite the foregoing, effective therapeutic methods are still needed.

### SUMMARY OF THE INVENTION

In accordance with one aspect of the instant invention, methods of treating, inhibiting, and/or preventing a muscular dystrophy in a subject are provided. The methods comprise administering acid sphingomyelinase to the subject. In a particular embodiment, the method comprises administering a nucleic acid encoding acid sphingomyelinase to the subject, particularly wherein the nucleic acid is expressed in the liver or hepatocytes. In certain embodiment, the nucleic acid encoding acid sphingomyelinase is under the control of or linked to a liver specific or hepatocyte specific promoter. Typically, the muscular dystrophy is a dysferlinopathy or is dysferlin deficient. Examples of dysferlinopathy include limb-girdle muscular dystrophy type 2B (LGMD2B) or Miyoshi muscular dystrophy 1. In a particular embodiment, the acid sphingomyelinase is human acid sphingomyelinase. In a particular embodiment, the nucleic acid encoding acid sphingomyelinase is contained within a viral vector, such as an adeno associated virus vector.

In accordance with another aspect of the instant invention, compositions and vectors (e.g., AAV vectors) for practicing the above methods are also provided.

### BRIEF DESCRIPTION OF THE DRAWINGS

FIG. 1A provides images of LGMD2B patient myoblasts treated with increasing doses of purified hASM protein. Confocal images of the myoblasts prior to and following focal laser injury (site marked by white arrow) showing FM dye labeling are provided. Scale bar = 10 µm. FIG. 1B provides a plot showing the kinetics of FM-dye entry into the myoblasts following membrane injury (n = 50 cells per condition). Data is presented as mean ± SEM. * p<0.05 (vs. Untreated and 3 U/L), # (vs. 5 U/L) by mixed model ANOVA with analyses for interaction effects between treatment condition and time. FIG. 1C provides a graph showing the quantification of the proportion of laser-injured cells that fail to repair (n > 45 cells per condition). Data is presented as mean ± SEM. 1-way ANOVA, with Tukey HSD post-hoc testing, with alpha set at p < 0.05 (n = 3 experimental repeats with 15-18 cells per repeat per condition).

FIG. 2A provides confocal images of the bottom surface (cell-coverslip interface) of mouse myoblasts expressing mRFP-tagged caveolin-1 either untreated (top) or treated with 6U/L purified hASM (bottom). Grayscale image shows the whole cell at the start of imaging (timepoint 0). The line on the cell marks the pixels shown in the grayscale kymograph demonstrating caveolin-1 mobility, presented as images acquired at 1 frame per second, for a 3-minute period (kymograph y-axis = total acquisition time of 180 seconds). The broken tracks of pixels indicates movement of caveolae present at the cell membrane. The arrow in the hASM-treated kymograph indicates the time of hASM addition at the 60-second mark.

FIG. 2B provides a plot showing quantification of caveolin-1 punctae in each condition (n = 50 puncate from 10 cells). FIG. 2C provides images of cell membrane shedding wherein live cells were labelled with FITC-cholesterol prior to imaging. Grayscale images show confocal image of the cell membrane at the coverslip surface at the start of imaging (timepoint 0), and the white box marks the extracellular space on the coverslip adjacent to the cell used to monitor the cholesterol-labelled vesicles shed by the cell. The zoom of this region is shown in the panels on the right wherein vesicles present at the onset of imaging (baseline), and vesicles present after 2-minutes after mock (untreated) treatment or treatment with 6 U/L hASM (hASM-treated) are indicated. FIG. 2D provides a graph showing the quantification of FITC-cholesterol-enriched particles shed by cells treated or not treated with 6U/L hASM (n =10 cells per condition). FIG. 2E provides a graph of the quantification of the rate of loss of cell associated FITC-cholesterol fluorescence by the cells imaged in FIGS. 2C and 2D (n=10 cells per condition). FIG. 2F provides images showing an optical section through the middle of mouse myoblasts expressing the CLIC/GEEC reporter GPI-GFP before and 4-minutes after treatment with 6U/L hASM. FIGS. 2G-2J provide plots showing kinetics (FIGS. 2G, 2I) and rate of internalization (FIGS. 2H, 2J) of GPI-GFP in C2C12-myoblasts (FIGS. 2G, 2H) and healthy and patient myoblasts (FIGS. 2I, 2J). Data represent mean ± SEM. *p < 0.05 (vs. Untreated cells) via independent sample t-test (FIGS. 2B, 2D, 2E). Kinetics and rate-analyses were performed via mixed model ANOVA, alpha set at p < 0.05 (FIGS. 2G-2J). Scale bars = 10 µm for whole cell and 5 µm for zoomed images.

FIG. 3A provides confocal images of mouse myoblast labeled with WGA at baseline and after 3 minutes of endocytosis in untreated and hASM-treated cells. Scale bar = 10 µm. FIG. 3B provides a plot showing the effect of different dose of hASM on bulk membrane endocytosis in mouse myoblasts. FIG. 3C provides confocal images showing fluorescent WGA-labelled healthy and patient myoblasts at baseline (left panel) and after the 3-minutes of endocytosis (right panel). Scale bar = 10 µm. FIG. 3D provides a plot showing quantification of bulk endocytosis by healthy and LGMD2B patient muscle cells and the effect of hASM on patient and healthy cell endocytosis (n > 2 experimental repeats per condition). All data are presented as mean ± SEM. FIGS. 3B, 3D: *p < 0.05 (vs. Untreated cells), assessed via 1-way ANOVA, and Tukey HSD post-hoc testing.

FIG. 4A provides an image of a Western blot for hASM in lysates from HepG2 cells transduced either with Control-AAV or hASM-AAV under a liver-specific promoter (n = 3 independent replicates). FIG. 4B provides a graph of the quantification of hASM activity in lysates from HepG2 cells transduced either with Control-AAV or hASM-AAV under a liver-specific promoter (n = 3 independent replicates). FIG. 4C provides a graph of the quantification of hASM activity in culture supernatants from control-AAV and hASM-AAV infected HepG2 cells. (n = 3 independent replicates). FIG. 4D provides confocal images of healthy and LGMD2B patient myoblasts prior to and following focal laser injury (site marked by arrow) showing FM dye labeling. LGMD2B patient myoblasts were treated with culture supernatants from control and hASM-AAV infected HepG2 cells in CIM. Scale bar = 10 µm. FIG. 4E provides a plot showing the averaged kinetics of FM-dye entry in healthy and patient myoblasts (n > 15 cells per condition). Data is presented as mean ± SEM. *p < 0.001 (vs. Control-AAV-Treated cells) by independent samples t-test (FIGS. 4B, 4C). For FIG. 4E, mixed model ANOVA with analyses for interaction effects between treatment condition and time was used (*p < 0.001, vs. Control-AAV-Treated cell supernatant).

FIG. 5A provides a plot showing hASM activity in the livers isolated from hASM-AAV and control-AAV injected mice (n = 5 mice per condition). FIG. 5B provides a plot showing hASM activity in the serum of hASM-AAV and control-AAV 12-weeks post injection (expressed in U/L). FIG. 5C provides a plot showing serum Alanine Transaminase (ALT) concentration to assess extent of liver damage in control- and hASM-AAV-treated mice 12-weeks after injection. FIG. 5D provides a graph showing the quantification of myofibers labeled with IgM. FIG. 5E provides images of myofibers labeled with IgM (arrowheads) (top row; scale bar=1000 µm); images of FM-dye uptake by myofibers in freshly isolated biceps, following focal laser injury at site marked by white arrow (n=20 myofibers per mouse) (middle two rows; scale bar=50 µm); and images of isolated EDL muscles from control-AAV and hASM-AAV treated mice injured by repeated 10% eccentric contractions and sarcolemma damage and labeled with procion orange dye (arrowheads) (bottom row; scale bar=100 µm. FIG. 5F provides a graph of the kinetics of FM-dye uptake by myofibers in freshly isolated biceps, following focal laser injury at site marked by white arrow (n=20 myofibers per mouse). FIG. 5G provides a plot showing the myofibers that successfully repaired from laser injury (n>15). FIG. 5H provides a graph showing quantification of the number of procion orange labeled fibers per muscle (n=4 muscle per group due to excess damage during preparation of EDL muscle in one mouse in each group). FIG. 5I provides a graph showing the change in muscle contractile force with 10 repeated eccentric contractions (n=5 mice per group). Data is presented as mean ± SEM. *p<0.05 vs. Control-AAV, independent samples t-test or mixed-model ANOVA with analyses for interaction effects between treatment condition and time. FIG. 5J provides a graph showing recombinant human ASM activity in 5 µg of muscle protein (quadriceps) (each sample analyzed in triplicate - Control-AAV, hASM-AAV, n = 5 mice per AAV group, and 3 per wild-type). Data is presented as mean hASM activity ± SEM. *p = 0.011 (vs. Control-AAV) by independent samples t-test.

FIG. 6A: Row 1 provides H&E-stained image of quadriceps muscle cross-sections of LGMD2B-model, 12-weeks after treatment with a single IV injection of control-AAV or hASM-AAV (arrowheads mark inflammatory foci) (Scale bar = 100 µm); Row 2 provides images showing muscle cross-sections labeled using Laminin and DAPI to visualize basement membrane and myonuclei, respectively (Scale bar = 100 µm); Row 3 provides images of Masson Trichrome staining in muscle cross-section (n = 5 per group) (Scale bar = 100 µm); and Row 4 provides images of Perilipin-labeled area in quadriceps muscle cross-section (Scale bar = 100 µm). FIG. 6B provides a plot showing quantification of inflammatory foci (10 randomly selected regions from entire quadriceps cross-sections from n = 5 mice per group). FIG. 6C provides a graph showing quantification of regenerated myofibers marked by the presence of central nuclei, across entire quadriceps cross-section, and expressed as % of total fibers. FIG. 6D provides a graph showing the distribution of myofiber cross-sectional areas (n=3000 fibers per group). FIG. 6E provides a graph showing the quantification of Masson Trichrome staining in muscle cross-section (n = 5 per group). FIG. 6F provides a graph of the quantification of Perilipin-labeled area in quadriceps muscle cross-section. FIGS. 6G and 6H provide graphs of the quantification of forelimb and hindlimb grip strength of mice treated as indicated and the contractile force is normalized to body weight (n = 5 mice per group, average of 5 repeats measure per mouse; Forelimb: p > 0.05). Data is presented as mean ± SEM. Hindlimb: *p<0.05 vs. Control-AAV.

FIG. 7A provides a plot showing paired changes in bodyweight of AAV-treated mice from prior-to and 12-weeks after single AAV injection. FIG. 7B provides a plot showing hASM activity in the serum of hASM-AAV and control-AAV injected mice at baseline (week 0), and at 1-, 4-, and 12-weeks post injection (expressed in U/L). Two 22-week-old wild-type BL6 mice were assessed for serum ASM activity as a comparison to normal levels (mean = dotted line, gray shading = min/max). *p = 0.011 (vs. Control-AAV). FIG. 7C provides a plot showing hASM cellular toxicity and concentration relationships. Human myoblasts were cultured in growth media supplemented with titrated concentrations of hASM protein (Control/PBS, 8, 80, 800 U/L) for 24 hours (6 U/L was the minimal therapeutic dose - denoted by the dashed line). Cells were collected and assessed for cell viability/death via trypan blue assay. Data is presented as mean proportion of cells that died (%) of total cell count. * p <0.001 (vs. 800 U/L hASM) by independent 1-way ANOVA. FIG. 7D provides a plot of histology scores for mice from each treatment group with suitable/quality histology images that afforded histopathological grading (scale: 1-10, with higher scores indicating worse pathology; 3 = focal degeneration/necrosis, 4 = focal/extensive inflammation/ degeneration/or necrosis, 5 = massive necrosis/hepatocyte loss; PBS injected BLA/J mouse score of 3: focal degeneration and necrosis). FIG. 7E provides a plot showing serum ALT levels across the 12-week study period, to assess the effects of the liver-targeted AAV on liver health and/or liver injury (normal range 5-month-old BL6 mice: ~22-40 U/L) (Otto, et al. (2016) J. Am. Assoc. Lab. Anim Sci., 55(4):375-86). Data is presented as mean ± SEM.

### DETAILED DESCRIPTION OF THE INVENTION

Herein, adeno associated virus (AAV) viral vectors were used to deliver the recombinant human acid sphingomyelinase gene (rhASM) specifically to the liver in mice. This rhASM-AAV may insert into the hepatocyte genome and induce the liver to upregulate its production of rhASM protein. Muscular dystrophies such as Limb-Girdle Muscular Dystrophy 2B (LGMD2B), characterized by a lack of dysferlin protein in skeletal muscle, suffer from poor repair of the damaged myofiber. The myofibers are frequently damaged during daily activity and muscle contraction, specifically at the muscle fiber membrane. Such membrane damage is normally efficiently repaired within minutes, due in part to dysferlin-mediated release of the reparative enzyme ASM. ASM released by the damaged muscle cell allows the enzyme to act on the damaged cell membrane where it hydrolyzes the lipid sphingomyelin and helps stabilize the membrane to promote repair of the injury (Defour et al. (2014) Cell Death Disease 5:e1306). However, lack of ASM or inhibition of its enzymatic action prevents the muscle fiber from repairing effectively and contributes to myofiber death and muscle degeneration. This deficit is an underlying mechanism for poor muscle health observed in LGMD2B and other muscular diseases.

One gene therapy approach for treating dysferlin deficit involves repeated delivery of AAVs encoding dysferlin directly to the muscles. This method is hampered by immune reactions and inflammatory responses mounted against the AAV vectors. Further, the large size of the dysferlin gene hampers or prevents its packaging into a single AAV vector, thereby reducing the efficacy of the treatment.

In contrast, the instant invention circumvents the need for repeated delivery of the dysferlin gene into the muscle. Indeed, by addressing the downstream consequence of dysferlin deficit — namely reduced ASM secretion leading to poor repair of the dysferlin deficient muscle fibers — and targeting rhASM-AAV to the liver, the instant invention provides unexpectedly superior and long term improvement in the repair capacity of dysferlinopathic myofibers and/or restoration of dysferlin. This invention thus represents a stable therapeutic approach to treat the poor myofiber repair ability in dysferlin deficient muscular dystrophies, particularly dysferliopathies such as LGMD2B. The present methods avoid the requirement for repeated administration of the vector and the difficulty associated with efficient delivery of AAV vectors into muscles.

Thus, the instant invention provides approaches to treating a dysferlin deficit by exogenous provision of ASM, particularly rhASM. The AAV-based gene therapy of the instant invention enables production and secretion of the ASM (e.g., rhASM) enzyme into circulation to increase the level of ASM (e.g., rhASM) enzymes in the skeletal muscles with dysferlin deficiency, which in turn aids in efficient repair of the diseased muscles. Using an AAV2/8-based vector, the rhASM gene was delivered into a mouse model (BL6/AJ) for dysferlin deficiency (LGMD2B) via tail vein injection and the efficacy of this approach for reducing deficits in the diseased mouse muscles was examined. Significantly, improvements in multiple facets of muscle health (contractile strength, histopathology, and repair capacity) were achieved to extents better than those reported for other methods.

In accordance with the instant invention, methods of treating, inhibiting, and/or preventing muscular dystrophies in a subject in need thereof are provided. In a particular embodiment, the muscular dystrophy is characterized by a dysferlin deficiency. In a particular embodiment, the muscular dystrophy is a dysferlinopathy. Examples of dysferlinopathies include, without limitation, Limb-Girdle Muscular Dystrophy 2B (LGMD2B) and Miyoshi Myopathy (MM) or Miyoshi muscular dystrophy 1.

In accordance with another aspect of the instant invention, methods of improving or increasing myofiber, particularly skeletal myofiber, repair are provided, particularly in a subject in need thereof. In a particular embodiment, the myofiber is characterized by a dysferlin deficiency. In a particular embodiment, the subject has a muscular dystrophy. In a particular embodiment, the subject has a dysferlinopathy. In a particular embodiment, the subject has Neimann Pick Disease (e.g., type A or B).

The methods of the instant invention comprise administering acid sphingomyelinase (ASM) to a subject in need thereof. In certain embodiments, the methods of the instant invention comprise administering a nucleic acid molecule encoding acid sphingomyelinase (ASM) to a subject in need thereof. In a particular embodiment, the ASM is human (hASM). In a particular embodiment, the ASM is a recombinant human ASM (rhASM) such as Olipudase alpha (Sanofi Pharmaceuticals, Bridgewater, NJ). GenBank Gene ID: 6609 and GenBank Accession Nos. NM_000543 and NP_000534 provide examples of amino acid and nucleotide sequences. The ASM can be any variant or isoform (e.g., isoform 1, 2, 3, 4, or 5) of ASM. In a particular embodiment, the ASM is isoform 1 or variant 1. In a particular embodiment, the ASM comprises the signal peptide.

An example of the precursor human ASM sequence with signal peptide is:

MPRYGASLRQSCPRSGREQGQDGTAGAPGLLWMGLVLALALALALALALS
DSRVLWAPAEAHPLSPQGHPARLHRIVPRLRDVFGWGNLTCPICKGLFTA
INLGLKKEPNVARVGSVAIKLCNLLKIAPPAVCQSIVHLFEDDMVEVWRR
SVLSPSEACGLLLGSTCGHWDIFSSWNISLPTVPKPPPKPPSPPAPGAPV
SRILFLTDLHWDHDYLEGTDPDCADPLCCRRGSGLPPASRPGAGYWGEYS
KCDLPLRTLESLLSGLGPAGPFDMVYWTGDIPAHDVWHQTRQDQLRALTT
VTALVRKFLGPVPVYPAVGNHESTPVNSFPPPFIEGNHSSRWLYEAMAKA
WEPWLPAEALRTLRIGGFYALSPYPGLRLISLNMNFCSRENFWLLINSTD
PAGQLQWLVGELQAAEDRGDKVHIIGHIPPGHCLKSWSWNYYRIVARYEN
TLAAQFFGHTHVDEFEVFYDEETLSRPLAVAFLAPSATTYIGLNPGYRVY
QIDGNYSGSSHVVLDHETYILNLTQANIPGAIPHWQLLYRARETYGLPNT
LPTAWHNLVYRMRGDMQLFQTFWFLYHKGHPPSEPCGTPCRLATLCAQLS
ARADSPALCRHLMPDGSLPEAQSLWPRPLFC (SEQ ID NO: 1).

An example of the mature human ASM sequence is:

LALSDSRVLWAPAEAHPLSPQGHPARLHRIVPRLRDVFGWGNLTCPICKG
LFTAINLGLKKEPNVARVGSVAIKLCNLLKIAPPAVCQSIVHLFEDDMVE
VWRRSVLSPSEACGLLLGSTCGHWDIFSSWNISLPTVPKPPPKPPSPPAP
GAPVSRILFLTDLHWDHDYLEGTDPDCADPLCCRRGSGLPPASRPGAGYW
GEYSKCDLPLRTLESLLSGLGPAGPFDMVYWTGDIPAHDVWHQTRQDQLR
ALTTVTALVRKFLGPVPVYPAVGNHESTPVNSFPPPFIEGNHSSRWLYEA
MAKAWEPWLPAEALRTLRIGGFYALSPYPGLRLISLNMNFCSRENFWLLI
NSTDPAGQLQWLVGELQAAEDRGDKVHIIGHIPPGHCLKSWSWNYYRIVA
RYENTLAAQFFGHTHVDEFEVFYDEETLSRPLAVAFLAPSATTYIGLNPG
YRVYQIDGNYSGSSHVVLDHETYILNLTQANIPGAIPHWQLLYRARETYG
LPNTLPTAWHNLVYRMRGDMQLFQTFWFLYHKGHPPSEPCGTPCRLATLC
AQLSARADSPALCRHLMPDGSLPEAQSLWPRPLFC (SEQ ID NO: 2)
.

An example of a nucleic acid sequence encoding human ASM sequence is:

agtcagccga ctacagagaa gggtaatcgg gtgtccccgg cgccgcccgg ggccctgagg
gctggctagg gtccaggccg ggggggacgg gacagacgaa ccagccccgt gtaggaagcg
cgacaatgcc ccgctacgga gcgtcactcc gccagagctg ccccaggtcc ggccgggagc
agggacaaga cgggaccgcc ggagcccccg gactcctttg gatgggcctg gtgctggcgc
tggcgctggc gctggcgctg gcgctggctc tgtctgactc tcgggttctc tgggctccgg
cagaggctca ccctctttct ccccaaggcc atcctgccag gttacatcgc atagtgcccc
ggctccgaga tgtctttggg tgggggaacc tcacctgccc aatctgcaaa ggtctattca
ccgccatcaa cctcgggctg aagaaggaac ccaatgtggc tcgcgtgggc tccgtggcca
tcaagctgtg caatctgctg aagatagcac cacctgccgt gtgccaatcc attgtccacc
tctttgagga tgacatggtg gaggtgtgga gacgctcagt gctgagccca tctgaggcct
gtggcctgct cctgggctcc acctgtgggc actgggacat tttctcatct tggaacatct
ctttgcctac tgtgccgaag ccgcccccca aaccccctag ccccccagcc ccaggtgccc
ctgtcagccg catcctcttc ctcactgacc tgcactggga tcatgactac ctggagggca
cggaccctga ctgtgcagac ccactgtgct gccgccgggg ttctggcctg ccgcccgcat
cccggccagg tgccggatac tggggcgaat acagcaagtg tgacctgccc ctgaggaccc
tggagagcct gttgagtggg ctgggcccag ccggcccttt tgatatggtg tactggacag
gagacatccc cgcacatgat gtctggcacc agactcgtca ggaccaactg cgggccctga
ccaccgtcac agcacttgtg aggaagttcc tggggccagt gccagtgtac cctgctgtgg
gtaaccatga aagcacacct gtcaatagct tccctccccc cttcattgag ggcaaccact
cctcccgctg gctctatgaa gcgatggcca aggcttggga gccctggctg cctgccgaag
ccctgcgcac cctcagaatt ggggggttct atgctctttc cccatacccc ggtctccgcc
tcatctctct caatatgaat ttttgttccc gtgagaactt ctggctcttg atcaactcca
cggatcccgc aggacagctc cagtggctgg tgggggagct tcaggctgct gaggatcgag
gagacaaagt gcatataatt ggccacattc ccccagggca ctgtctgaag agctggagct
ggaattatta ccgaattgta gccaggtatg agaacaccct ggctgctcag ttctttggcc
acactcatgt ggatgaattt gaggtcttct atgatgaaga gactctgagc cggccgctgg
ctgtagcctt cctggcaccc agtgcaacta cctacatcgg ccttaatcct ggttaccgtg
tgtaccaaat agatggaaac tactccggga gctctcacgt ggtcctggac catgagacct
acatcctgaa tctgacccag gcaaacatac cgggagccat accgcactgg cagcttctct
acagggctcg agaaacctat gggctgccca acacactgcc taccgcctgg cacaacctgg
tatatcgcat gcggggcgac atgcaacttt tccagacctt ctggtttctc taccataagg
gccacccacc ctcggagccc tgtggcacgc cctgccgtct ggctactctt tgtgcccagc
tctctgcccg tgctgacagc cctgctctgt gccgccacct gatgccagat gggagcctcc
cagaggccca gagcctgtgg ccaaggccac tgttttgcta gggccccagg gcccacattt
gggaaagttc ttgatgtagg aaagggtgaa aaagcccaaa tgctgctgtg gttcaaccag
gcaagatcat ccggtgaaag aaccagtccc tgggccccaa ggatgccggg gaaacaggac
cttctccttt cctggagctg gtttagctgg atatgggagg gggtttggct gcctgtgccc
aggagctaga ctgccttgag gctgctgtcc tttcacagcc atggagtaga ggcctaagtt
gacactgccc tgggcagaca agacaggagc tgtcgcccca ggcctgtgct gcccagccag
gaaccctgta ctgctgctgc gacctgatgc tgccagtctg ttaaaataaa gataagagac
ttggactcca (SEQ ID NO: 3).

In a particular embodiment, the nucleic acid sequence encoding human ASM sequence comprises the portion of SEQ ID NO: 3 from the start codon to the stop codon (indicated by underlining above).

The ASM of the instant invention may have an amino acid sequence having at least 80%, at least 85%, at least 90%, at least 95%, at least 97%, at least 99%, or 100% identity with SEQ ID NO: 1 or 2.

In a particular embodiment, the nucleic acid molecule encoding acid sphingomyelinase is under the control of a liver-specific or hepatocyte specific protomer (Jacobs et al. (2008) Gene Ther., 15(8):594-603; Kramer et al. (2003) Mol. Ther., 7(3):375-385). Liver-specific or hepatocyte specific protomers preferentially express the linked nucleic acids in liver cells or hepatocytes over other cell types or tissues. Liver-specific or hepatocyte specific protomers need not — but may —exclusively express the linked nucleic acid in liver cells or hepatocytes. Examples of liver-specific or hepatocyte specific protomers include, without limitation, human α-1 antitrypsin (hAAT) promoter, hybrid liver promoter (HLP; McIntosh, et al. (2013) Blood 121(17):3335-44), human thyroxine-binding globulin (TBG), human serum albumin promoter (optionally linked to one or more copies of the human prothrombin enhancer), DC190 promoter (Ziegler, et al. (2004) Mol. Ther., 9:231-240). In a particular embodiment, liver-specific or hepatocyte specific protomer is the human serum albumin promoter or the DC190 promoter.

In a particular embodiment, the nucleic acid molecule encoding acid sphingomyelinase is delivered (e.g., passively (e.g., intravenously) or directly (e.g., injection)) to the liver. In certain embodiments, the nucleic acid molecule encoding acid sphingomyelinase is not directly delivered (e.g., by injection) to the muscle of the subject.

In a particular embodiment, the nucleic acid molecule encoding acid sphingomyelinase is contained within a plasmid or a vector (e.g., expression vector), particularly a viral vector. The nucleic acid molecules of the invention may optionally be contained in or encapsulated by non-viral vectors (e.g., liposomes, micelles, naked cDNA, transposons, etc.). Viral vectors which may be used in the present invention include, but are not limited to, adenoviral vectors, adeno-associated virus (AAV) vectors (e.g., AAV-1 to AAV-13, particularly AAV-2, AAV-5, AAV-7, and AAV-8, or hybrid AAV vectors), lentiviral vectors and pseudo-typed lentiviral vectors, herpes simplex virus vectors, vaccinia virus vectors, and retroviral vectors. In a particular embodiment, the nucleic acid molecule encoding acid sphingomyelinase is contained within an AAV vector. In a particular embodiment, the AAV vector is an AAV2/8 vector or AAV8 vector. In a particular embodiment, the vector or viral vector is targeted to the liver or hepatocytes (e.g., with a targeting ligand or a liver- or hepatocyte-specific receptor ligand). In a particular embodiment, the nucleic acid molecule encoding acid sphingomyelinase is under the control of a liver-specific or hepatocyte specific protomer as explained above.

The nucleic acid molecules encoding acid sphingomyelinase or vectors comprising the same of the instant invention (or compositions comprising the same with a pharmaceutically acceptable carrier) can be administered to an animal, in particular a mammal, more particularly a human, in order to treat, inhibit, or prevent a muscular dystrophy. The methods and compositions of the instant invention may also comprise at least one other therapeutic agent for treating, inhibiting, or preventing the muscular dystrophy (e.g., protein ASM). The additional therapeutic agent may also be administered in a separate composition from the compounds of the instant invention. The compositions may be administered at the same time and/or at different times (e.g., sequentially).

The compounds of the instant invention described herein will generally be administered to a patient or subject as a pharmaceutical preparation. The term “patient” as used herein refers to human or animal subjects. The compounds of the instant invention may be employed therapeutically, under the guidance of a physician or other healthcare professional.

The pharmaceutical preparation comprising the nucleic acid molecules encoding acid sphingomyelinase or vectors comprising the same of the invention may be conveniently formulated for administration with an acceptable medium such as water, buffered saline, ethanol, polyol (for example, glycerol, propylene glycol, liquid polyethylene glycol and the like), dimethyl sulfoxide (DMSO), oils, detergents, suspending agents, or suitable mixtures thereof. Solubility limits may be easily determined by one skilled in the art.

As used herein, “pharmaceutically acceptable medium” or “carrier” includes any and all solvents, dispersion media and the like which may be appropriate for the desired route of administration of the pharmaceutical preparation, as exemplified in the preceding discussion. The use of such media for pharmaceutically active substances is known in the art. Except insofar as any conventional media or agent is incompatible with the compounds to be administered, its use in the pharmaceutical preparation is contemplated.

The dose and dosage regimen of the compounds according to the invention that is suitable for administration to a particular patient may be determined by a physician considering the patient’s age, sex, weight, general medical condition, and the specific condition for which the compounds are being administered and the severity thereof. The healthcare provider may also take into account the route of administration of the compounds, the pharmaceutical carrier within which the compounds are contained, and the compound’s biological activity.

Selection of a suitable pharmaceutical preparation will also depend upon the mode of administration chosen (e.g., into the bloodstream, intravenously or direct injection). For example, the nucleic acid molecules encoding acid sphingomyelinase or vectors comprising the same of the instant invention may be administered by injection, e.g., directly into or near the liver. In these instances, the pharmaceutical preparation comprises the compounds of the invention dispersed in a medium that is compatible with the site of injection.

Nucleic acid molecules encoding acid sphingomyelinase or vectors comprising the same of the instant invention may be administered by any method such as intranasal, intramuscular, subcutaneous, topical, oral, or injection. Pharmaceutical preparations for injection are known in the art. If injection is selected as a method for administering the compounds, steps should be taken to ensure that sufficient amounts of the compounds reach their target cells to exert a biological effect.

Pharmaceutical compositions containing the compounds of the present invention as the active ingredient in intimate admixture with a pharmaceutical carrier can be prepared according to conventional pharmaceutical compounding techniques. The carrier may take a wide variety of forms depending on the form of preparation desired for administration, e.g., injection. Injectable suspensions may be prepared, for example, using appropriate liquid carriers, suspending agents, and the like.

### Definitions

The following definitions are provided to facilitate an understanding of the present invention:

The singular forms “a,” “an,” and “the” include plural referents unless the context clearly dictates otherwise.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., polysorbate 80), emulsifier, buffer (e.g., Tris HCl, acetate, phosphate), antimicrobial, bulking substance (e.g., lactose, mannitol), excipient, auxiliary agent, or vehicle with which an active agent of the present invention is administered. Pharmaceutically acceptable carriers can be sterile liquids, such as water and oils, including those of petroleum, animal, vegetable, or synthetic origin. Water or aqueous saline solutions and aqueous dextrose and glycerol solutions may be employed as carriers, particularly for injectable solutions. Suitable pharmaceutical carriers are described in “Remington’s Pharmaceutical Sciences” by E.W. Martin (Mack Publishing Co., Easton, PA); Gennaro, A. R., Remington: The Science and Practice of Pharmacy, (Lippincott, Williams and Wilkins); Liberman, et al., Eds., Pharmaceutical Dosage Forms, Marcel Decker, New York, N.Y.; and Kibbe, et al., Eds., Handbook of Pharmaceutical Excipients, American Pharmaceutical Association, Washington.

The term “treat” as used herein refers to any type of treatment that imparts a benefit to a patient afflicted with a disease, including improvement in the condition of the patient (e.g., in one or more symptoms), delay in the progression of the condition, etc.

As used herein, the term “prevent” refers to the prophylactic treatment of a subject who is at risk of developing a condition resulting in a decrease in the probability that the subject will develop the condition.

As used herein, the term “subject” refers to an animal, particularly a mammal, particularly a human.

A “therapeutically effective amount” of a compound or a pharmaceutical composition refers to an amount effective to prevent, inhibit, treat, or lessen the symptoms of a particular disorder or disease. The treatment of a disease or disorder herein may refer to curing, relieving, and/or preventing the disease or disorder, the symptom(s) of it, or the predisposition towards it.

As used herein, the term “therapeutic agent” refers to a chemical compound or biological molecule including, without limitation, nucleic acids, peptides, proteins, and antibodies that can be used to treat a condition, disease, or disorder or reduce the symptoms of the condition, disease, or disorder.

A “vector” is a genetic element, such as a plasmid, cosmid, bacmid, phage or virus, to which another genetic sequence or element (either DNA or RNA) may be attached so as to bring about the replication and/or expression of the attached sequence or element. A vector may be either RNA or DNA and may be single or double stranded. An “expression vector” is a specialized vector that contains a gene or nucleic acid sequence with the necessary regulatory regions (e.g., promoter) needed for expression in a host cell.

The term “linked” or “operably linked” means that the regulatory sequences necessary for expression of a coding sequence are placed in the nucleic acid molecule in the appropriate positions relative to the coding sequence so as to effect expression of the coding sequence. This same definition is sometimes applied to the arrangement of coding sequences and transcription control elements (e.g. promoters, enhancers, and termination elements) in an expression vector or recombinant vector.

The following example describes illustrative methods of practicing the instant invention and is not intended to limit the scope of the invention in any way.

### EXAMPLE

Skeletal muscle cells, or myofibers, enable physical movement and are frequently damaged by strenuous activity, overload and eccentric contractions (McNeil, et al. (2003) Annu. Rev. Cell Dev. Biol., 19:697-731; Horn, et al. (2018) Cellular Molecular Life Sci., 75(20):3751-70). Mutations that increase myofiber fragility or impede repair result in muscle degeneration and muscular dystrophies (Wallace, et al. (2009) Annu. Rev. Physiol., 71:37-57). Miyoshi Myopathy (MM) and Limb-Girdle Muscular Dystrophy 2B (LGMD2B) are two such autosomal recessive muscular dystrophies that manifest in early adulthood and lead to progressive skeletal muscle weakness and wasting (Aoki, M., In: Adam et al., eds., GeneReviews, Seattle, WA, 1993). These diseases (collectively called dysferlinopathies) are caused by mutations in the DYSF gene, which encodes a large (237 kDa) muscle membrane protein - dysferlin (Liu, et al. (1998) Nat. Genet., 20(1):31-6; Bashir, et al. (1998) Nat. Genet., 20(1):37-42). Even prior to overt muscle degeneration, dysferlinopathic patient myofibers exhibit plasma membrane (sarcolemma) defects including membrane tears, extrusions, sub-sarcolemmal accumulation of vesicles and vacuoles, and thickening of the basal lamina (Selcen, et al. (2001) Neurology 56(11):1472-81). Poor repair of sarcolemmal injury contribute to these early abnormalities (Selcen, et al. (2001) Neurology 56(11):1472-81; Cenacchi, et al. (2005) J. Clin. Pathol., 58(2):190-5). Damage to the myofiber sarcolemma is repaired by a complex multi-step process activated by the injury-triggered influx of extracellular calcium, which is compromised by dysferlin deficit (Bansal, et al. (2003) Nature 423(6936):168-72; Defour, et al. (2014) Cell Death Dis., 5:e1306). Failed or deficient myofiber repair activates chronic inflammatory responses and leads to muscle degeneration - a notable feature of dysferlinopathic skeletal muscle (Nagaraju, et al. (2008) Am. J. Pathol., 172(3):774-85; Gallardo, et al. (2001) Neurology 57(11):2136-8; Hogarth, et al. (2019) Nature Comm., 10(1):2430).

Repair of plasma membrane injury involves calcium-triggered signaling and vesicle fusion and fission, which is facilitated by calcium binding proteins including synaptotagmins (Bansal, et al. (2003) Nature 423(6936):168-72; Sonder, et al. (2019) Sci. Rep., 9(1):6726; Horn, et al. (2019) Curr. Top. Membr., 84:67-98; Horn, et al. (2017) Sci. Signal., 10(495):eaaj 1978; Sreetama, et al. (2016) Cell Death Differ., 23(4):596-607; Scheffer, et al. (2014) Nat. Commun., 5:5646; Jaiswal, et al. (2014) Nat. Commun., 5:3795; Bittel, et al. (2019) Front. Phys.,10:828; Jaiswal, et al. (2002) J. Cell Biol., 159(4):625-35; Jaiswal, et al. (2004) PLoS Biol., 2(8):e233). Similar to synaptotagmins, dysferlin is a member of the C2 domain protein family, which includes proteins that bind negatively-charged membrane phospholipids in a calcium-dependent manner (Rizo, et al. (1998) J. Biol. Chem., 273(26):15879-82; Lek, et al. (2012) Traffic 13(2):185-94). Dysferlin mediates sarcolemmal repair by tethering lysosomes to the plasma membrane, facilitating lysosomes to exocytose immediately following membrane injury (Defour, et al. (2014) Cell Death Dis., 5:e1306). Rapid lysosomal exocytosis allows the lysosomal enzyme ASM to be secreted within seconds of sarcolemmal injury — a process required for repair (Tam, et al. (2010) J. Cell Biol., 189(6): 1027-38; Michailowsky, et al. (2019) Skelet. Muscle 9(1):1). Lack of dysferlin, delays and reduces injury-triggered lysosome exocytosis, thereby slowing and reducing ASM secretion upon cell injury (Defour, et al. (2014) Cell Death Dis., 5:e1306). Consequentially, reduced ASM secretion in injured LGMD2B cells or lack of ASM production in Niemann-Pick disease type A (NPDA) cells, compromises myofiber sarcolemmal repair (Defour, et al. (2014) Cell Death Dis., 5:e1306; Michailowsky, et al. (2019) Skelet. Muscle 9(1):1). These deficits identify extracellular ASM supplementation as a potential treatment to improve myofiber repair for both LGMD2B and NPDA patients.

Upon secretion into the extracellular medium, ASM hydrolyzes sphingomyelin lipids within the plasma membrane to ceramide, which is proposed to remove damaged portions of the plasma membrane through extracellular vesicle (ECV) shedding and by endocytosis (Tam, et al. (2010) J. Cell Biol., 189(6):1027-38; Bianco, et al. (2009) EMBO J., 28(8):1043-54). Plasma membrane injured by pore forming toxins has been found to undergo both ECV shedding and caveolar endocytosis (Keyel, et al. (2011) J. Cell Sci., 124(Pt 14):2414-23; Corrotte, et al. (2013) Elife 2:e00926), with these toxins also colocalizing with Glycosylphosphotidylinositol (GPI) — the marker of endosomes formed by clathrin-independent carriers (CLIC) (Idone, et al. (2008) J. Cell. Biol., 180(5):905-14; Mayor, et al. (2014) Cold Spring Harb. Perspect. Biol., 6(6): a016758). However, details of how ASM helps repair physiological (focal or mechanical) injury to the plasma membrane were unresolved. Understanding the role of ASM in repair of physiological membrane injury is important for informing treatments for muscle, lung, and other diseases involving cell membrane injuries.

Pre-clinical gene therapy approaches for LGMD2B aiming to re-express the skeletal muscle dysferlin gene have resulted in a mixed, but overall positive therapeutic outlook (Potter, et al. (2018) Hum. Gene Ther., 29(7):749-62; Pryadkina, et al. (2015) Mol. Ther. Methods Clin. Dev., 2:15009; Lostal, et al. (2012) PLoS One, 7(5):e38036). The progress of these therapies to the clinic, however, requires overcoming barriers associated with the efficient packaging and muscle delivery of large genes such as dysferlin (Bulaklak, et al. (2017) Curr. Opin. Pharmacol., 34:56-63). Drug based therapies offer an alternative, but currently there are no approved drugs to address poor repair, or other disease etiology of dysferlinopathy. However, preclinical studies indicate that drugs that stabilize the sarcolemma can enhance myofiber repair and improve dysferlinopathic muscle function (Sreetama, et al. (2018) Molecular Therapy, 26(9):2231-42; Gushchina, et al. (2017) Mol. Ther., 25(10):2360-71). Extracellular ASM improves dysferlinopathic myofiber repair (Defour, et al. (2014) Cell Death Dis., 5:e1306). Intravenous delivery of hASM has shown efficacy (Miranda, et al. (2000) FASEB J., 14(13):1988-95; Murray, et al. (2015) Mol. Genet. Metab., 114(2):217-25; Samaranch, et al. (2019) Sci. Transl. Med., 11(506):eaat3738; Dodge, et al. (2005) Proc. Natl. Acad. Sci., 102(49):17822-7), and clinical safety of hASM for treating NPDA (Wasserstein, et al. (2019) Mol. Genet. Metab., 126(2):98-105; Wasserstein, et al. (2018) J. Inherit. Metab. Dis., 41(5):829-38). However, utility of this approach for treating LGMD2B, or improving skeletal muscle deficits in NPDA has not been tested. Here, the pre-clinical efficacy is examined of hASM protein and a non-muscle targeted AAV-based gene therapy approach to improve sarcolemmal repair in LGMD2B using patient muscle cell and mouse models. The LGMD2B mouse model was also used to examine the use of hASM-AAV gene therapy for chronic improvement of myofiber repair, muscle histopathology, and muscle function.

### Materials and Methods

### Animals

B6.A-Dysf^{prmd}/GeneJ (B6A/J) mice were purchased from the Jackson Laboratory (Bar Harbor, ME) and maintained in the animal house of the Children’s Research Institute (CRI). All experiments involving the use of mice were approved by the CRI animal care and use committee. Animals were housed in a germ-free facility under a controlled 12 hours light/dark cycle with free access to food and water. Animals were genotyped before using in the experiment.

### Cell Culture and Treatments

Immortalized control (Healthy donor) and LGMD2B patient (with homozygous c.4882G mutation, leading to loss of any detectable dysferlin protein) myoblasts were used as described (Defour, et al. (2014) Cell Death Dis., 5:e1306). Myoblasts were cultured in human myoblast culture media kit (Promocell), supplemented with 10% FBS, on 0.4% gelatin coated dishes and maintained at 37° C. and 5% CO₂. HepG2 and C2C12 myoblast line were cultured in high-glucose DMEM supplemented with 10% FBS, and 1% Penicillin/Streptomycin. For laser injury, cells were plated on fibronectin-coated glass coverslips. The cells were either injured as such or pre-incubated in cell imaging media (CIM: HBSS with 10 mM HEPES, 1 mM calcium-chloride, pH 7.4), for 20 minutes with varying concentrations of purified hASM (R&D Systems, Minneapolis, MN), or in culture supernatant of HepG2 cells transduced with hASM-AAV or control (eGFP-AAV) viral particles. The cells were laser-injured in CIM containing 1 µg/µl cell impermeant dye FM1-43 (N-(3-Triethylammoniumpropyl)-4-(4-(Dibutylamino) Styryl) Pyridinium Dibromide; Life Technologies) and the same concentrations of hASM and cell supernatant as in the incubation period. Injury and subsequent imaging were performed at 37° C. in the stage-top ZILCS incubator (Tokai Hit Co., Fujinomiya-shi, Japan). 1- to 5-µm² area of plasma membrane was irradiated for <10 ms with a pulsed laser (Ablate!™, 3i Intelligent Imaging Innovations, Inc. Denver, CO) and cells were imaged at 2 second intervals with a 60X/1.45 NA oil objective on an IX81 Olympus microscope (Olympus America, Center Valley, PA) equipped with a diode laser of 488 nm (Cobolt, Sweden). FM dye intensity (F/F₀ where F₀ is the original intensity) was quantified and repair was indicated by the block of FM entry leading to increase in FM dye fluorescence as described (Defour, et al. (2014) J. Vis. Exp., 2014(85):e51106).

### Endocytosis Assays

For bulk endocytosis cell membrane of myoblasts (~70% confluent) were labeled with AF488-conjugated wheat germ agglutinin (WGA) (3 ug/mL) for 2 minutes at 37° C. After washing the excess WGA with CIM cells were left untreated or treated with hASM (6 U/L in CIM), and imaged using 40X/1.4 NA or 60X/1.45 NA oil objective on an IX81 Olympus microscope (Olympus America, Center Valley, PA), simultaneously in widefield and confocal modes. WGA endocytosis was allowed and at different time points bromophenol blue (BPB) was injected in the imaging chamber (final concentration of 4 mM) to quench WGA at the cell surface. To assess the extent of membrane endocytosis, following background correction, average post-quench fluorescence of each cell was divided by its initial pre-quench fluorescence, and normalized to the fraction of internalized membrane assessed after immediate quenching (0-min endocytosis).

For caveolar endocytosis cells transfected with mRFP-tagged caveolin-1 were imaged as described (Tagawa, et al. (2005) J. Cell Biol., 170(5):769-79). Cells were imaged in CIM with a 60X/1.45 NA oil objective as described above, using an IX81 Olympus microscope (Olympus America, Center Valley, PA) equipped using a confocal diode laser of 560 nm (Cobolt, Sweden), at the membrane-coverslip interface. Cells were imaged at 1 Hz as indicated. To quantify caveolin mobility, 50 individual caveolin puncta/vesicles were marked in each cell at the start of imaging. Each vesicle was subsequently tracked manually. A vesicle was deemed mobile if it either migrated laterally for a distance >1.5 µm or moved axially such that it was absent from the imaging plane for > 10 seconds, or both. Fraction of vesicles (out of 50 for each cell) were quantified for the 2-minute.

For CLIC/GEEC endocytosis assay, cells were transfected with glycosylphosphatidylinositol tagged with GFP (GPI-GFP) (Nichols, et al. (2001) J. Cell Biol., 153(3):529-41). Transfected cells were imaged as above at a z-plane through the mid of the cell body at 1 frame/minute for 20-minutes. As needed, hASM was added to the chamber after the 2nd image. GPI-GFP membrane fluorescence was monitored by marking cell membrane and corrected for photobleaching. Endocytosis rates were obtained by curve fitting the membrane fluorescence kinetics trace spanning the timepoint of interest and using this to calculate the rate of loss of membrane fluorescence at that specific timepoint. Images were quantified using SlideBook™ 6.0 (Intelligent Imaging Innovations, Inc, Denver CO).

### Membrane Shedding Assay

C2C12 cells (at ~50% confluence), were labeled with FITC-PEG-Cholesterol (5 µM; PEG-2000, Nanocs Inc., PG2-CSFC-2k) for 30 minutes, at 37° C. in CIM. After washing the excess label cells were immediately imaged in CIM by simultaneous confocal and widefield microscopy, with a 60X/1.45 NA oil objective on IX81 microscopy equipped with a diode laser of 488 nm. Cells were imaged at 0.2 Hz, for 2-minutes. As needed, hASM was added ~20-30 seconds prior to onset of time-lapse acquisition. The images were collected at z-plane positioned at the cell-coverslip interface to monitor vesicle shed on the surrounding coverslip area. Vesicles were quantified using Metamorph 7.0 (Molecular Devices, CA) in a 5,000 µm² area on the coverslip surface adjacent to the cell (sum of vesicles shed over the 2-minute period) and normalized to vesicles present at the onset of acquisition. To assess the loss of cellular fluorescence widefield images were corrected for photobleaching, followed by analysis of the loss of fluorescence in 2-minute period, using SlideBook™ 6.0 software.

### Western Blotting and Immunostaining

HepG2 Cell lysate were resolved in 4-12% gradient polyacrylamide gel, transferred to nitrocellulose membranes, and probed with the indicated antibodies against: ASM (Abcam, Cambridge, MA) and β-actin (Abcam, Cambridge, MA). Primary antibodies were followed by the appropriate HRP-conjugated secondary antibodies (Sigma-Aldrich), and chemiluminescent western blotting substrate (GE Healthcare, Pittsburgh, PA) and processed on Chemidoc™ MP system (BioRad Laboratories, CA).

### AAV Vector Generation and Delivery

For AAV8/DC190-hASM vector production, a previral plasmid carrying human ASM cDNA was constructed (Barbon, et al. (2005) Mol. Ther., 12(3):431-40). Briefly, expression of the human acid sphingomyelinase cDNA (NM_000543) is driven from the liver-restricted promoter/enhancer DC190 (Ziegler, et al. (2004) Mol. Ther., 9:231-240; human serum albumin promoter linked to two copies of the human prothrombin enhancers). The expression cassette also contains a hybrid intron. The polyadenylation signal is followed by a fragment of the human α1-antitrypsin intron, bringing the size of the recombinant viral DNA to approximately 4.5 Kb for optimal packaging. Plasmid DNA was purified using a Qiagen EndoFree® Plasmid purification kit (Germantown, MD). The AAV2-based pre-viral plasmid was packaged onto AAV serotype 8 capsids. Recombinant AAV virus was produced by triple plasmid transfection followed by cesium chloride density gradient purification by the University of Massachusetts Medical School Vector Core Gene Therapy Center (Worcester, MA). Genome copy titers of the AAV vectors were determined using a real-time TaqMan® PCR assay (ABI Prism 7700; Applied Biosystems, Foster City, CA) with primers that were specific for the bovine growth hormone polyadenylation signal sequence. AAV9.CMV.PI.eGFP.WPRE. bGH (Lot # CS0273) was used as the control AAV vector (Vector core at the Perelman School of Medicine, University of Pennsylvania). Viral particles were stored as suspension in sterile PBS with 5 % glycerol buffer at -80° C. The viral particle suspension was thawed, diluted and delivered via intravenous administration of with a viral dose of 3.4×10¹¹ particles per mouse or 1.1 × 10¹³ vg/kg. Mice used for this study were derived from two separate litters of BLA/J mice consisting of a mixture of male and female mice that were born on the same day. Each pup was identified by ear-tag ID, and a random draw from each litter was based on coded ID numbers to ensure - 1) Mix of mice from both litters were allocated to each treatment group, 2) Both male and female mice were represented in each treatment group. In the hASM-AAV group 5 mice were injected with hASM-AAV. Control group having same number of mice was injected with control AAVs. After the injection, experimental mice were kept in the home cage for 3 months and subjected to the specific experimentation.

### ASM Measurement

Livers and quadriceps muscle were snap frozen in liquid-nitrogen cooled isopentane (and stored at -80° C.), while serum - collected via retro-orbital bleeding at baseline, 1-, 4- and 12-weeks post injection, was stored in -80° C. For assays, tissue samples were ground and homogenized with a microtube homogenizer in RIPA buffer (Sigma-Aldrich, St. Louis MO) + protease inhibitor cocktail (Fisher Scientific, Waltham, MA) on ice. Lysates were assessed for total protein concentration using a BCA protein assay and plate-reader. Equal amounts of total lysate protein (4.1 µg for liver, 25 µg for quadriceps muscle), and serum volume (5 µL) were used across all samples. As ASM protein undergoes post-translational modifications, which affect the enzymatic activity, instead of protein amount the hASM activity was measured using Amplex™ Red Sphingomyelinase assay kit (Invitrogen). All samples were run in triplicate. Activity was thus expressed as units of hydrolytic activity (U) per gram of liver and muscle tissue (for liver/muscle ASM activity), and U per liter of serum. Activity was averaged across the 5 samples per treatment condition and expressed as mean + SEM.

### Serum Alanine Transaminase (ALT) Concentration

Serum (5 µL) from each of the above-listed timepoints post-AAV-injection, was assayed for ALT concentration — a marker of liver damage/disease, using a colorimetric assay (Cayman Chemical, Ann Arbor, MI) according to the manufacturer’s instructions. All samples were run in triplicate, with the ALT concentration averaged across all samples per treatment condition, per timepoint, and expressed as mean ± SEM.

### hASM-AAV Mediated in Vitro hASM Production and Quantification

HepG2 cells in a 96 well dish at a density of ~1×10⁵ cells/well were infected in antibiotic-free DMEM with 4.5×10⁶ particles of Ad5 (multiplicity of infection (MOI) of 45 pts/cell) for 2 hours. Cells were infected with AAV2/8 DC190-hASM or control vector at 1×10¹⁰ genome copies/ml (MOI of 10⁴) in a volume of 100 µl for 1 hour. After 1 hour, 100 µl of complete DMEM was added. On day 5, the cell culture media was collected and used immediately for subsequent experiments or stored in -80° C. Cells were pelleted by scraping in ice-cooled phosphate buffered saline and lysed with RIPA buffer (Sigma-Aldrich) containing protease inhibitor cocktail (Fisher Scientific, Waltham, MA). The culture supernatant and cell lysates were used for fluorimetric assay of hASM activity measurement using the Amplex™ Red Sphingomyelinase assay kit (Invitrogen) and for western blots. ASM kinetics was analyzed over the course of 20 minutes using EnSpire® Multimode Plate Reader (PerkinElmer) with fluorescence emission detection at 585 nm. hASM activity was thus expressed in units of activity per L of supernatant, or gram of cell lysate. All samples were assessed in triplicate and standard curve was generated. ASM activity of hASM was transposed from fluorescence emission values to units of activity using the known activity and fluorescence emission of the bacterial sphingomyelinase positive control (10 U/L) and the generated standard curve shown here. hASM protein has a units-of-ASM-activity conversion of 0.01 units per mg protein.

### hASM Cellular Toxicity in Vitro

Healthy donor myoblasts were cultured in 0.4% gelatin-coated 51 cm culture dishes, and were grown to 60% confluence in human myoblast culture media kit (Promocell), supplemented with 10% FBS, and maintained at 37° C. and 5% CO₂. Upon reaching 60% confluence, growth media was supplemented with titrated concentrations of hASM protein (Control/PBS, 8, 80, 800 U/L) for 24 hours. Subsequently, cells were collected and assessed for cell viability/death via trypan blue assay, with cell death expressed as a percentage of total cells. Cell death experiments were conducted with 3 biological replicates per hASM dosage.

### Histology and Immunohistochemistry

8-µm thick transverse cryosections of the quadriceps muscle and liver were prepared using CM3050S cryostat (Leica Biosystems, Buffalo Grove, IL), and stored at -20° C. for later staining (n=5 per group). After thawing, muscle sections were processed for H&E, Laminin (1:100, Anti-Laminin-2 alpha-chain, Rat monoclonal, Sigma-Aldrich), IgM (1:100, Invitrogen), Perilipin (1:250, Sigma), Masson trichrome (Trichrome Stain Kit, Abcam, Cambridge, MA), while liver sections were processed for H&E only. Images were captured with a VS120 slide scanning microscope (Olympus America, MA) at 40x magnification, and quantified using CellSens software. For immunostaining, muscle sections were blocked in 5% BSA for 1 hour (laminin-staining) or 1% BSA, 10% goat serum and 0.1%Tween (for perilipin). Alexa Fluor® 488 or 594 (1:500) secondary antibodies were used and co-stained with WGA and DAPI.

To quantify muscle inflammation, clusters of extramyofibrillar nuclei consisting of >9 nuclei were noted as inflammatory foci and quantified from 10 randomly chosen areas of the entire quadriceps cross-section in H&E-stained sections, and expressed per mm2 cross-sectional area. These sections were also used to quantify central-nucleated fibers, which was expressed as a percentage of total myofibers counted per muscle section. Centrally nucleated myofibers counts were independently verified using Laminin and DAPI-co-stained sections, and the CellProfiler Muscle Analyzer pipeline as described (Lau, et al. (2018) Skelet. Muscle 8(1):32.). Same pipeline was used to assess myofiber cross-sectional area across 3 mice per group, for a total of 3000 fibers per group, and measured in µm². Muscle fibrosis/collagen accumulation was quantified using Masson’s Trichrome staining. 5 representative images per quadriceps cross-section were taken from the whole muscle image and assessed for percentage of total muscle area taken up by stained collagen tissue (stained blue), using ImageJ as described (Corbiere, ET AL. (2018) J. Funct. Morphol. Kinesiol., 3(1):1). Selected images were split into red, blue, and green channels, with subsequent thresholding for the blue channel image to quantify collagen-stained fibrotic tissue.

For quantification of in vivo injured myofibers, the total number of wheat germ agglutinin (WGA)-labeled fibers from randomly chosen areas of entire quadriceps cross-sections was scored for fibers that were positive for IgM. These were then presented as the number of IgM-positive fibers across 1 mm² cross-sectional area of the muscle. To quantify adipogenic deposits, perilipin-stained quadriceps muscle sections were measured using Metamorph® software and presented as percentage of perilipin-positive area. For the liver histopathology scoring, H&E-stained sections were scored for features such as hepatocyte necrosis, apoptosis, karyolysis, degeneration, loss (focal or diffuse), vacuolation, hypertrophy, fibrosis, and inflammation on a scale of 1-5 (higher scores indicating worse pathology). Each liver sample score was average of score from 5 representative fields per liver section.

### Grip-Strength Measurement

Forelimb and hindlimb grip-strength measurement (GSM) were assessed using a grip strength meter (Columbus Instruments, Columbus, OH) as described (Spurney, et al. (2009) Muscle Nerve 39(5):591-602). The animals were acclimatized for 3 days before data collection. The forelimb and hindlimb grip-strength data were then collected over 5 consecutive days and represented as averaged grip strength/kg body weight over 5 days as described (Sreetama, et al. (2018) Molecular Therapy 26(9):2231-42).

### Ex Vivo Myofiber Injury

For contraction-induced sarcolemmal injury, EDL muscles were extracted from wild-type BL6 or from B6A/J mice treated with hASM-AAV or control-AAV, and placed in Ringer’s solution (137 mM NaCl, 24 mM NaHCO₃, 11 mM glucose, 5 mM KCl, 2 mM CaCl₂, 1 mM MgSO₄, 1 mM NaH₂PO₄, and 0.025 mM tubocurarine chloride) bubbled with 95% O₂ - 5% CO₂ to maintain pH at 7.4. The distal tendon was securely connected to a fixed bottom plate, and the proximal tendon was attached to the arm of a servomotor (800A in vitro muscle apparatus, Aurora Scientific) with 6-0 silk sutures. The vertically aligned EDL muscle was flanked by two stainless steel plate electrodes. Using single 0.2-mm square simulation pulses, the muscle was adjusted to the optimal muscle length for force generation. At optimal length, with isometric tetanic contractions 300 ms in duration at frequencies up to 250 Hz separated by 2 minutes of rest intervals, the maximal force was determined. Contraction-induced sarcolemma damage was induced by nine sequential lengthening contractions (LCs) with 10% strain at a velocity of two fiber lengths per second. Each contraction was separated by a 1-minute rest interval. LC-induced force loss was expressed as percentage of first contraction. At the end of LC protocol, muscles were trimmed of tendons, blotted, weighed, and incubated in a 0.2% PO solution at room temperature for 30 minutes. After washing the excess dye, the tissue was snap frozen in liquid-nitrogen-cooled isopentane prior to being sectioned and imaged for PO-labeled fibers, with unlabeled tissue being used to determine background fluorescence. The number of PO-positive myofibers was expressed as a percentage relative to the total myofibers in the muscle cross-section and fibers at the edge of the sections were excluded from analysis. For focal laser injury assay, intact biceps muscles were mounted in pre-warmed tyrodes buffer (119 mM NaCL, 5 mM KCL, 25 mM Hepes buffer, 2 mM CaCl₂, 2 mM MgCl₂, glucose - 6 g/L, pH 7.4), with FM 1-43 dye (1-2 mg/mL) and imaged using the 40X/1.4 NA the IX81 Olympus microscope as described for cell laser injuries above. Repair kinetics and successful myofiber repair determined as described for cell injury assays (Horn, et al. (2017) Sci Signal, 10(495):eaaj 1978).

### Study Rigor

A priori sample size determination for the in vivo portion of this study was derived from two studies conducted assessing the pro-reparative effect of membrane lipid stabilizing drugs (bacterial sphingomyelinase, and Vamorolone) (Defour et al. (2014) Cell Death Dis., 5:e1306; Sreetama, et al. (2018) Molecular Therapy 26(9):2231-42). For laser ablation injury assessment of repair capacity, a power analysis was performed from the Vamorolone trials, finding an effect size of 0.725 with this membrane lipid-modifying drug. With a two-tailed alpha set at 0.05, and power at 80%, this dictates that 5 mice per treatment group are required to achieve statistical significance. Similarly, bacterial sphingomyelinase improved myofiber membrane repair capacity with an effect size of 0.6, requiring use of 6 mice per group to assess significant effect on repair capacity assuming two-tailed alpha of 0.05, and power at 80%. Thus, upon compiling this data from studies examining the effects of compounds or drugs that modify cell membrane lipids in LGMD2B (BLA/J mice), as hASM does, 5 mice per group were required for the primary endpoint measure (membrane repair capacity) and 4-7 mice per treatment group to find statistically significant differences for additional end points tested.

All in vivo measures (laser injury assays, all muscle and liver histology measures, ASM and ALT activity, eccentric force assay) were obtained by a blinded member of the research team. Blinding was accomplished through the use of a deidentifying code sheet, that contained mouse ear-tag number and treatment group. The repair assays were coded to blind the rater/data analyzer, to condition. Assays involving added recombinant hASM were conducted by an unblinded team member, but the rater was blind to sample identity for in vitro ASM activity assays.

### Statistics

For cell-injury and biceps myofiber repair kinetics (FM-dye-intensity kinetics), eccentric force decrement traces, and CLIC-GEEC endocytosis kinetics, all generated curves were compared via mixed model ANOVA with analyses for interaction effects between the main effects of treatment condition and time or trial. In the event of significant interaction, group differences in FM dye fluorescence intensity/membrane fluorescence/eccentric force, was assessed per time point via Holm-Sidak test, and Huynh-Feldt correction due to violation of sphericity. One-way ANOVA was used to determine differences in the number of cells and/or myofibers that failed to repair following injury, and in general membrane endocytosis measures. Repeated-measures ANOVA was used to assess for differences in bodyweight changes over the 12-week treatment period, and in CLIC/GEEC endocytosis rates, between conditions. Comparisons between Control-AAV and hASM-treated mice in - hepatic ASM production, serum ASM activity, serum ALT concentration, proportion of fibers that repair with injury, histology measures (IgM+ proportion, Mason trichrome staining for fibrotic area, inflammatory foci, central nucleation, perilipin+ proportion, procion-orange+ proportion, and myofiber area), and limb force measurements, were calculated using independent samples t-test. Similarly, independent samples t-tests were used to calculate differences in ASM activity of transfected HepG2 cells (both cell supernatants and lysates), and in C2C12 caveolin endocytosis mobile fraction, and membrane shedding measure (Untreated vs. hASM-Treated). For all statistical analysis, alpha level was set at p < 0.05.

### Results

### hASM Restores LGMD2B Patient Cell Repair Independent of Vesicle Shedding and Caveolar Endocytosis

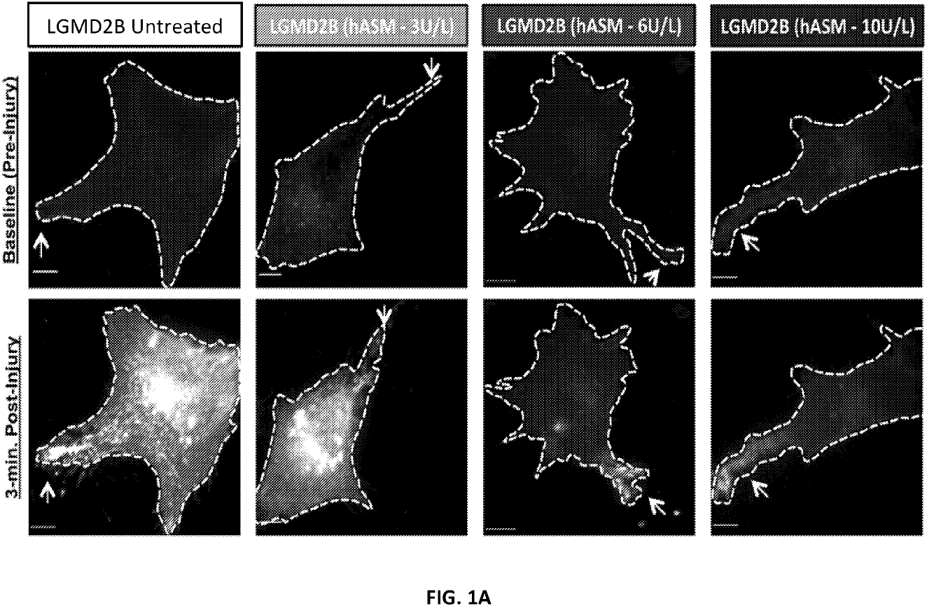

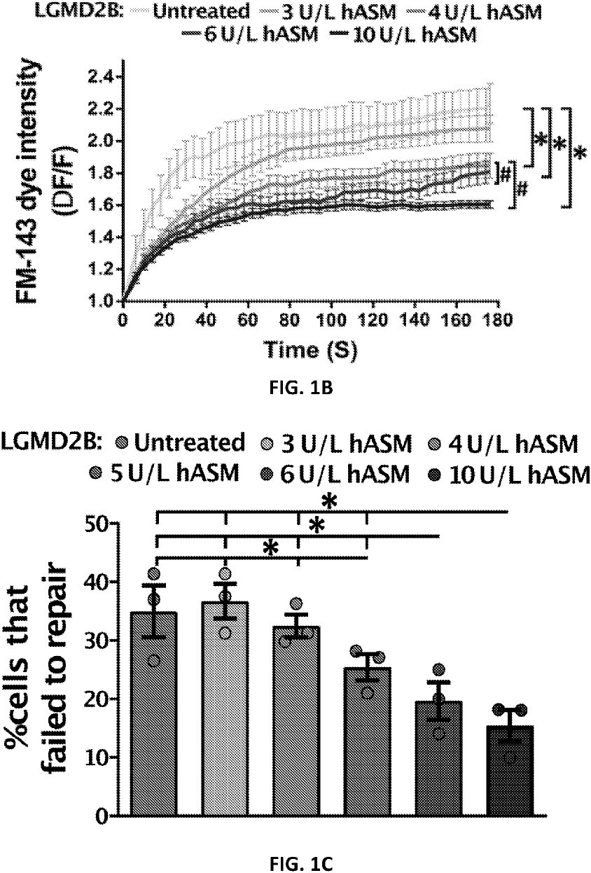

To test the effect of hASM on membrane repair, myoblasts from LGMD2B patients were treated with purified human ASM (hASM) protein. Exposure of patient cells to purified hASM caused a dose-dependent improvement in their plasma membrane repair (FIGS. 1A-1C). Purified hASM, at a concentration of 3 U/L, or 4 U/L, was not efficacious in improving repair of patient myoblasts (FIGS. 1A, 1B). However, treating the patient cells with purified hASM doses above 4 U/L improved cell membrane repair, reducing FM dye entry in the injured cells (FIGS. 1A, 1B). A clear dose-response of hASM effect on patient cell membrane repair emerges such that membrane repair was improved at the dose of 5 U/L hASM, and the peaked at the hASM concentrations of 6 U/L or above (FIGS. 1B, 1C). Consequently, while at 5U/L of hASM reduced the number of cells that failed to repair from injury, greatest improvement was attained at the hASM dose of 6 U/L and above (FIG. 1C).

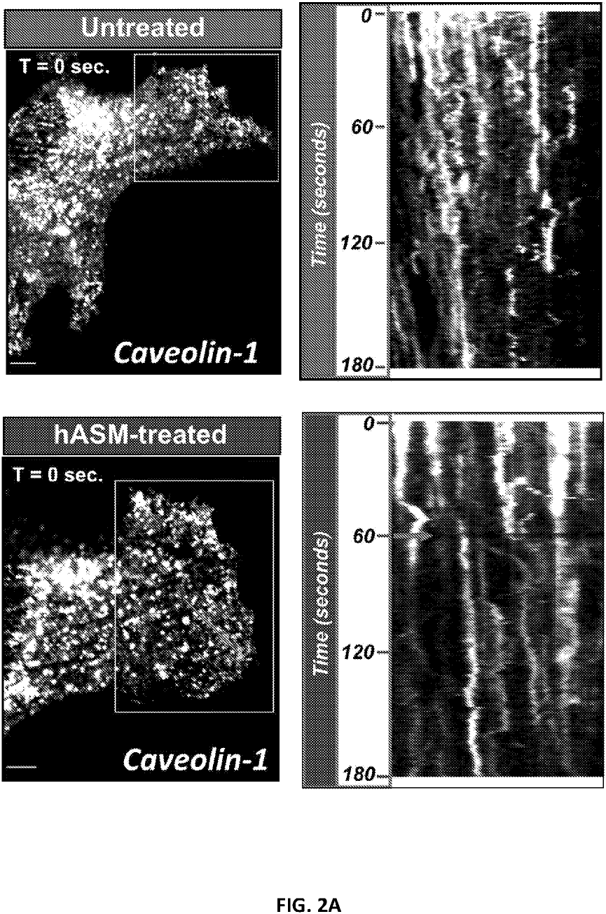

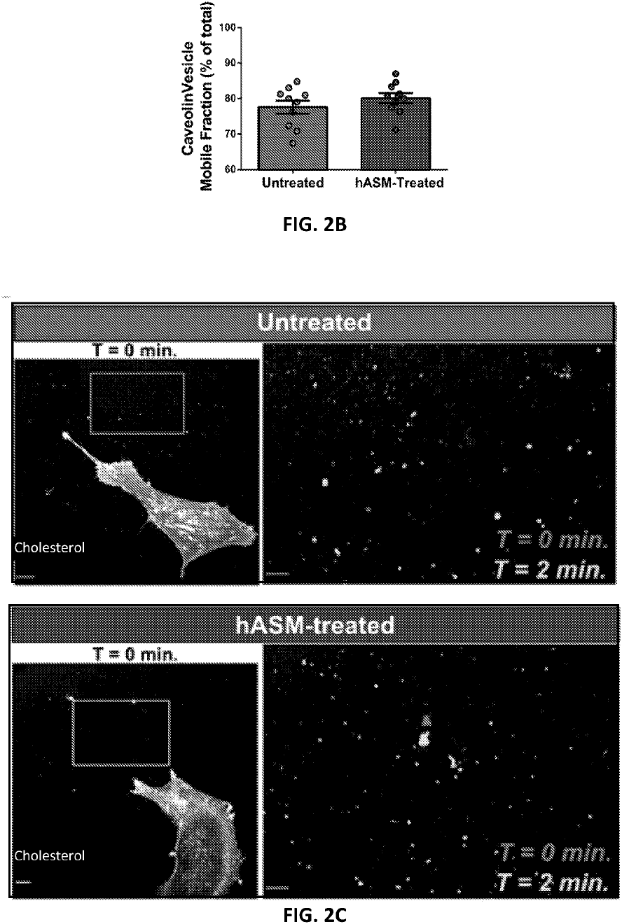

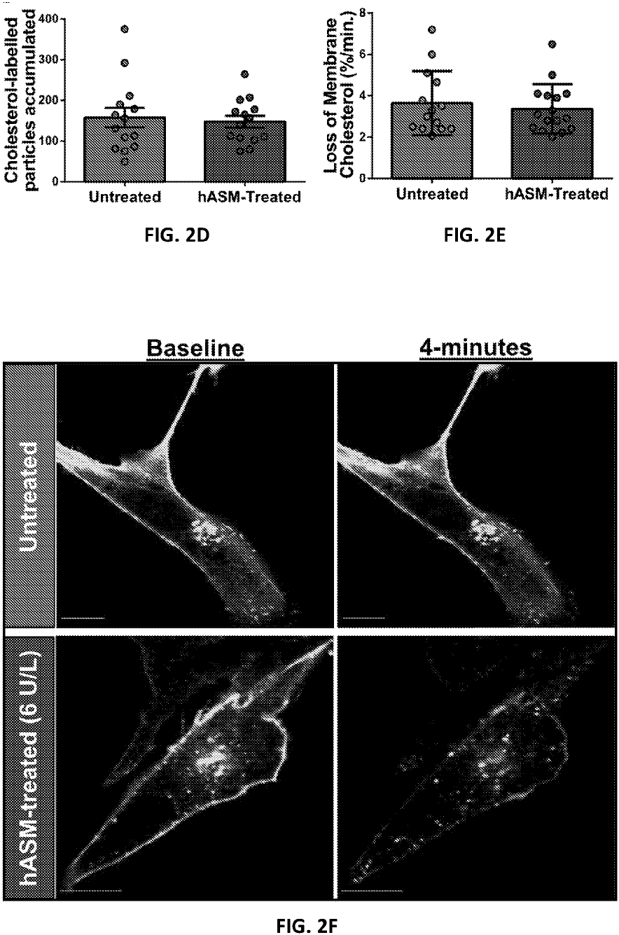

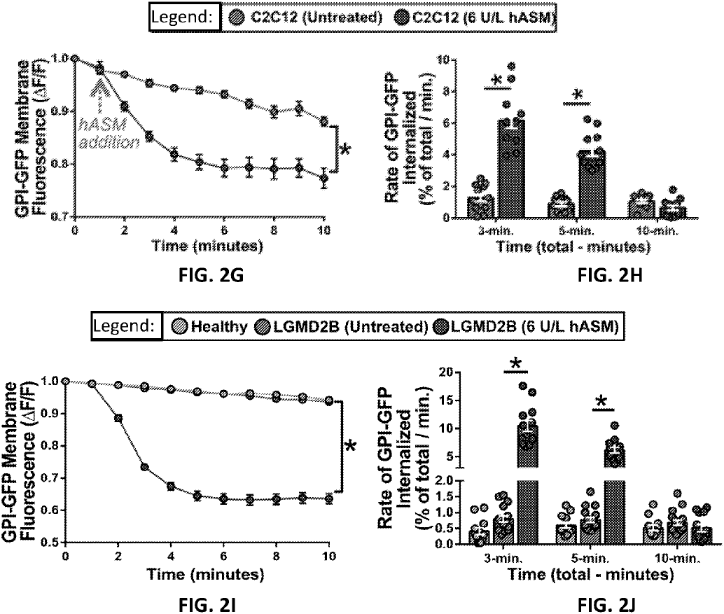

With the involvement of caveolar endocytosis and membrane shedding in repairing membrane injury by pore forming toxins, the effect of hASM on these pathways was examined. Caveolar endocytosis was monitored by live imaging caveolae dynamics in myoblasts expressing caveolinl-RFP (FIG. 2A). Imaging individual plasma membrane-associated caveolae by confocal microscopy showed that within a 2-minute period over 75% of caveolae at the plasma membrane moved from their starting position, and treatment with 6U/L of purified hASM did not affect this fraction of mobile caveolae (78% ± 1.8 vs. 80% ± 1.4; FIGS. 2A, 2B). Next, it was examined if this dose of hASM triggers plasma membrane shedding. As ECVs are enriched in cholesterol (Bittel, et al. (2019) Front. Physiology 10:828), the cell membrane was labelled with FITC-labeled PEG cholesterol and vesicle shedding was quantified over a 2-minute period (FIG. 2C). Untreated, and 6 U/L hASM-treated cells shed similar number of ECVs - Untreated: 158 ± 24 vs. hASM-treated: 147±15 (FIG. 2D) and led to the loss of similar amounts of cell-associated cholesterol labeling in hASM treated and untreated cells (FIG. 2E). These results determined that the dose of hASM which improves membrane repair in patient cells did not enhance caveolar endocytosis or ECV shedding, alluding to the existence of alternate membrane trafficking mechanisms by which hASM improves cell membrane repair.

### hASM Treatment Enhances Bulk Plasma Membrane Endocytosis

As a cell’s bulk endocytosis is supported by the clathrin independent carriers (CLICs), and CLICs facilitate endocytosis of dysferlin and pore-forming toxins (Idone, et al. (2008) J. Cell. Biol., 180(5):905-14; Hemandez-Deviez, et al. (2008) J. Biol. Chem., 283(10):6476-88), the effect of hASM treatment on CLIC marker Glycosyl-phosphatidylinositol (GPI)-anchored Green Fluorescent Protein (GPI-GFP) was examined. Using C2C12 myoblasts, a steady endocytosis of CLICs from the plasma membrane was observed, which was acutely enhanced by treatment with hASM (FIGS. 2F-2H). A similar rate of CLIC endocytosis was observed in untreated healthy and LGMD2B patient myoblasts (FIGS. 2F, 2I, 2J). Similar to the increase in CLIC endocytic rate in mouse myoblasts (FIG. 2H), hASM treatment of patient myoblasts also increased the CLIC endocytic rate (FIGS. 2I, 2J).

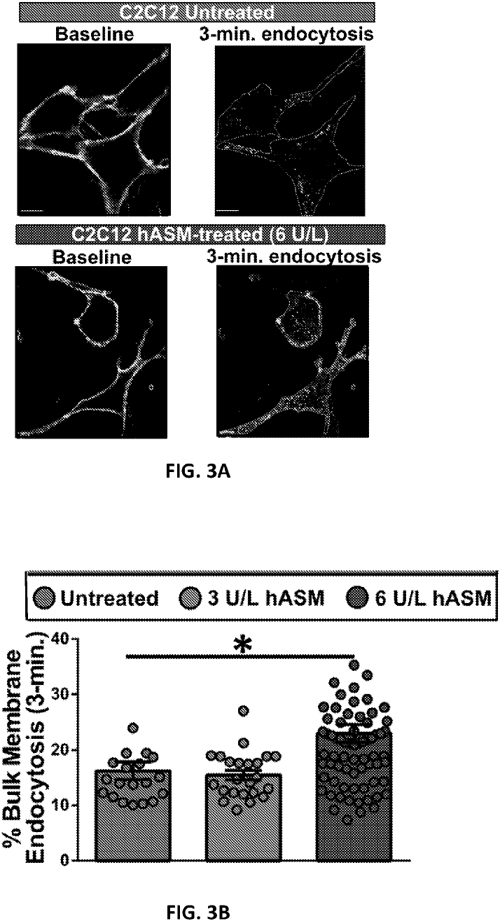

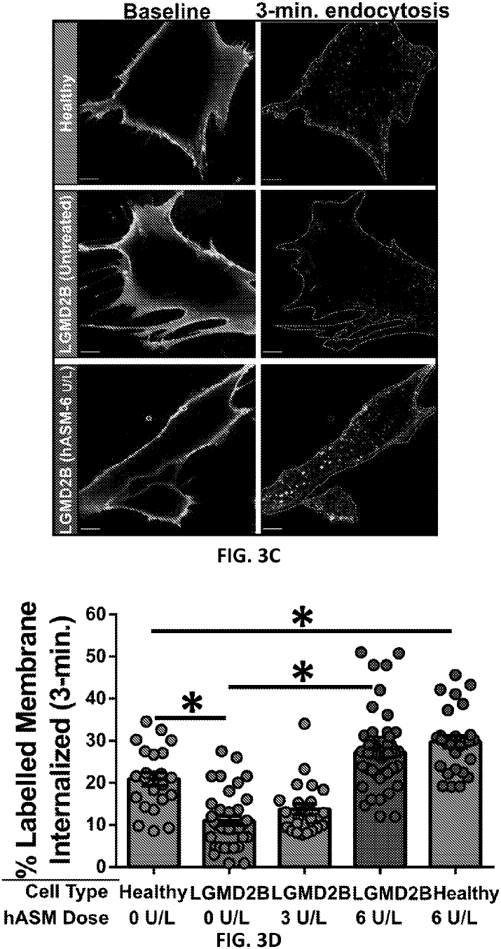

With the role of CLICs in bulk membrane removal, the role of bulk membrane endocytosis in repair was examined by using the lectin wheat germ agglutinin (WGA) to label the plasma membrane and assess its endocytic removal in response to different doses of hASM. Briefly, cell membrane was labelled with fluorescent WGA and membrane endocytosis was monitored over a 3-minute period by quenching the WGA fluorescence at the cell surface by using bromophenol-blue (BPB) at the end of endocytosis period. Punctate fluorescence in the cell, not quenched by BPB, marks the internalized WGA localized in endosomes. Internalized WGA fluorescence was expressed relative to the baseline labelling prior to quenching. Untreated mouse myoblasts and those treated with 3U/L hASM endocytosed similar amounts of the plasma membrane-associated WGA, but treatment with 6U/L hASM significantly increased the rate of WGA endocytosis in mouse muscle cells (Untreated: 16.2% + 1.6, 3 U/L hASM: 15.5% + 0.9, 6 U/L hASM: 23% + 1.6, WGA internalized) (FIGS. 3A, 3B).

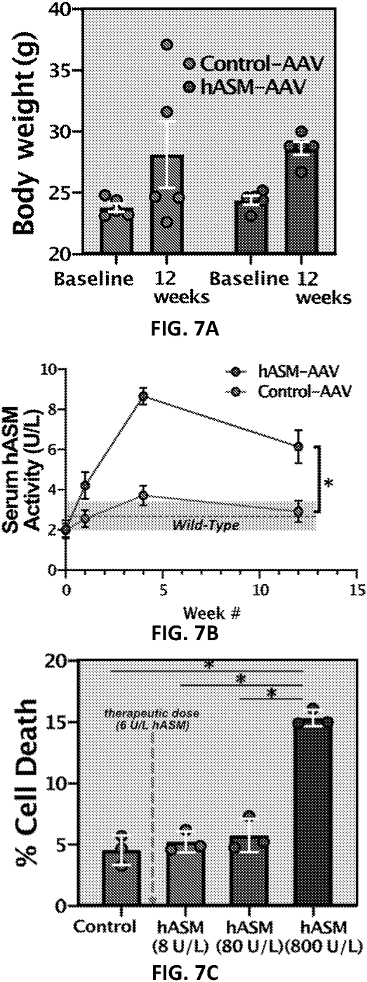

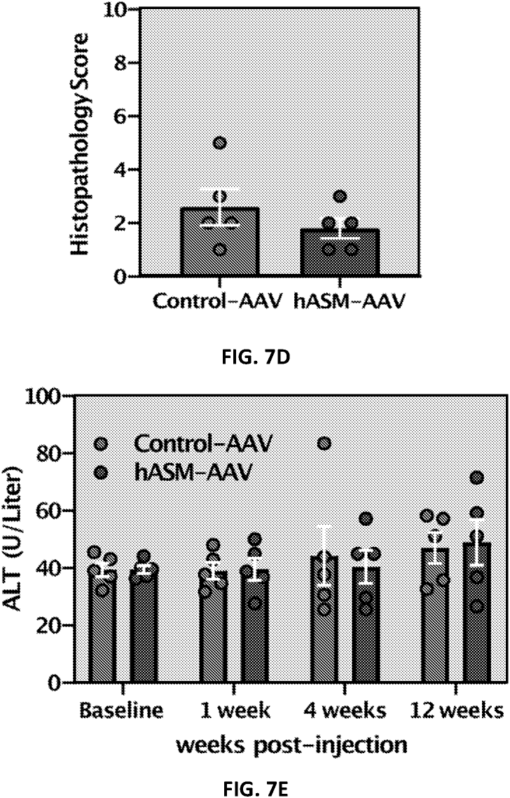

In accordance with the findings of reduced ASM secretion by LGMD2B patient myoblasts (Defour, et al. (2014) Cell Death Dis., 5:e1306), these cells exhibited 2-fold reduction in their ability to endocytose WGA (11% + 1 patient vs. 21% + 1.6 healthy) (FIGS. 3C, 3D). Treatment with the hASM dose that improved LGMD2B cell repair (6U/L), also enhanced WGA endocytosis of these cells, while the lower dose (3 U/L) failed to do so (FIGS. 3C, 3D). Treating healthy muscle cells with 6U/L hASM also increased WGA endocytosis by them (Untreated: 20.9% +1.6, 6 U/L hASM: 29.7% + 1.4) (FIG. 3D), without causing any cellular toxicity (FIG. 7). These findings identify that hASM-mediated improvement in repair safely enhances bulk plasma membrane endocytosis.

### hASM-AAV Offers a Genetic Approach to Restore Membrane Repair in LGMD2B

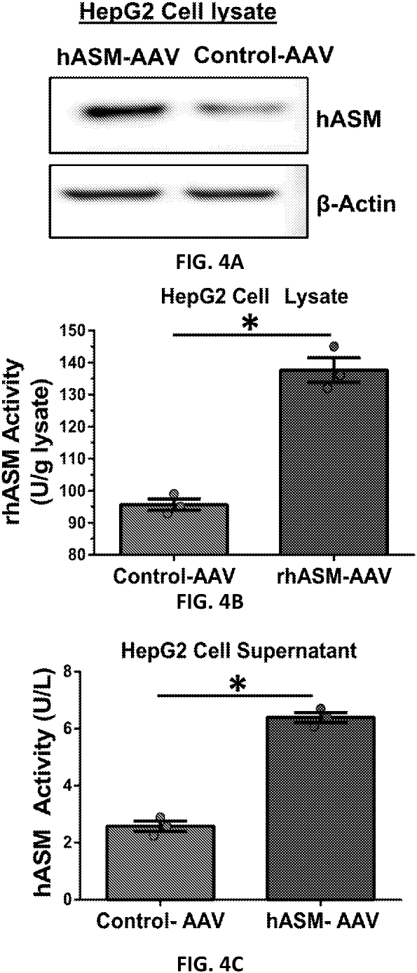

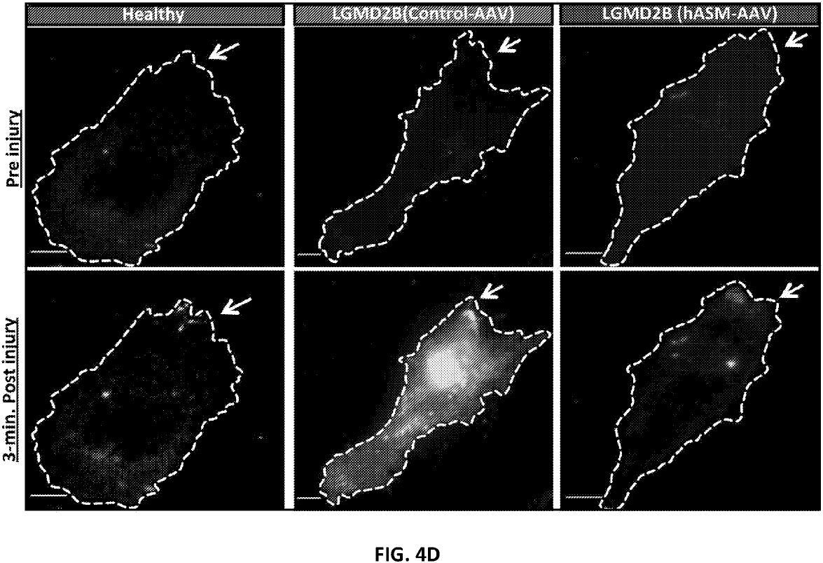

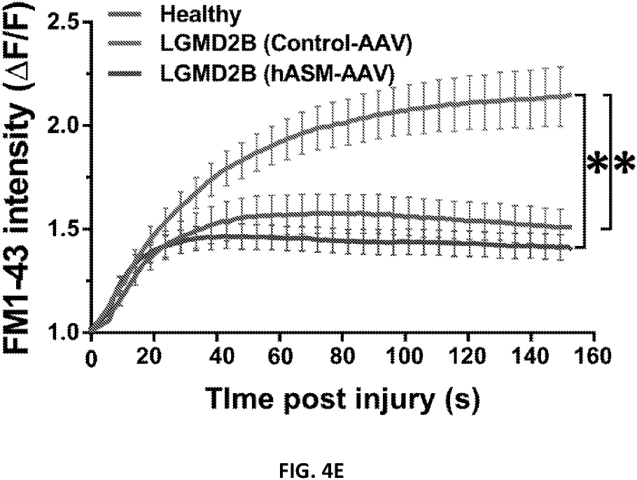

While the above studies demonstrate the utility of hASM treatment to safely address the bulk endocytosis defect in the LGMD2B patient cells, for its therapeutic utility the protein will require frequent administration to maintain a therapeutic level in vivo. To overcome this challenge, the use of an alternate approach was explored by genetically expressing secreted hASM to maintain a stable therapeutic level of this protein in the serum. An adeno-associated-virus (AAV) vector was used to express the secreted form of hASM protein under the control of a liver-specific promoter. An AAV vector was assessed in vitro by infecting the human liver cell line HepG2 with hASM-AAV that produces secreted hASM under a liver-specific promoter (Barbon, et al. (2005) Mol. Ther., 12(3):431-40). Compared to the control-vector, HepG2 cells infected with hASM-AAV secreted 6.4 U/L hASM, (FIGS. 4A-4C). As this is above therapeutic dose needed to improve membrane repair (6 U/L), the ability of secreted hASM produced by the human liver cells to improve repair of injured LGMD2B patient muscle cells was tested. Compared to the patient myoblasts treated with the culture supernatant from control-AAV expressing HepG2 cells, patient myoblasts treated with the supernatant of hASM-expressing HepG2 cells repaired efficiently, with kinetics similar to the healthy donor myoblasts (FIGS. 4D, 4E). These findings established the in vitro efficacy of liver-targeted hASM-AAV gene therapy to improve plasma membrane repair in LGMD2B patient muscle cells.

### Myofiber Sarcolemmal Repair Is Improved by Liver Targeted hASM-AAV

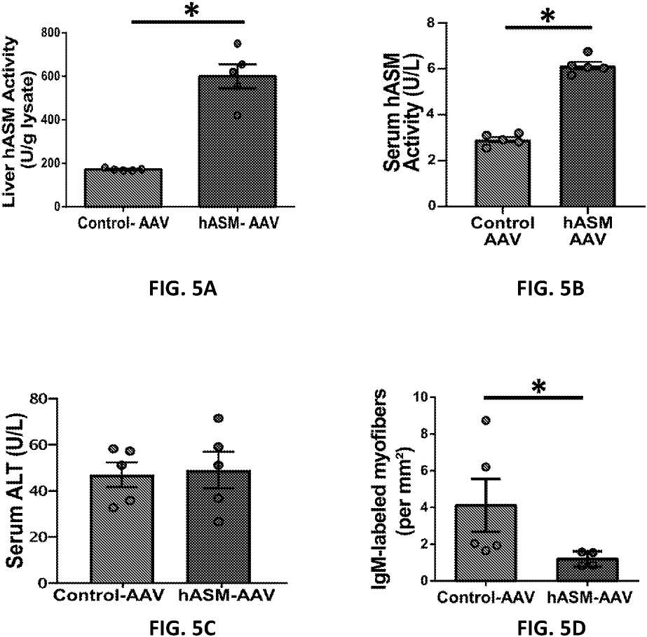

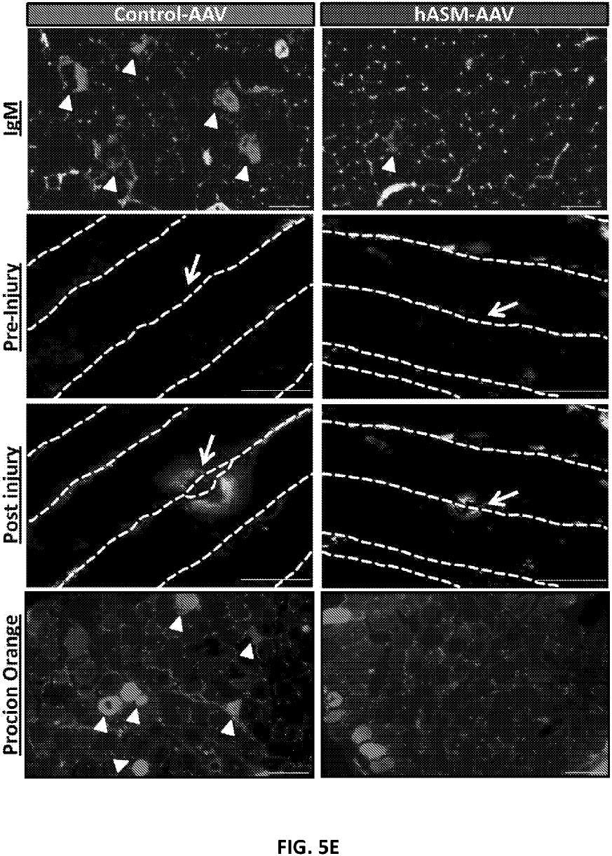

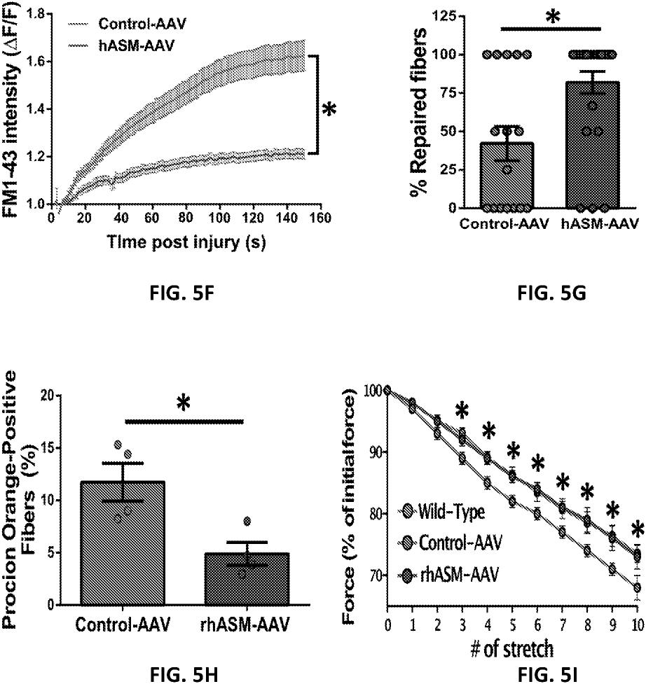

To test the in vivo efficacy of hASM in improving plasma membrane repair in LGMD2B muscle fibers, a mouse model of LGMD2B (B6A/J) was used. These dysferlin-deficient mice were treated once with liver-specific hASM-AAV or Control-AAV at 10 weeks of age by tail-vein injection. 12-weeks after this single dose of hASM-AAV, these mice were assessed at the age of 22 weeks. By 15-24 weeks of age, B6A/J mice show signs of muscle damage, myofiber repair deficit, and locomotor deficits, which continue to worsen progressively (Defour, et al. (2014) Cell Death Dis., 5:e1306; Hogarth, et al. (2019) Nature Communications 10(1):2430; Nagy, et al. (2017) Physiol Rep., 5(6):e13173). In mice treated with hASM-AAV there was a 4-fold higher liver hASM activity and 2-fold higher serum ASM activity as compared to those treated with control-AAV (600 + 54.7 U/gram v/s 171.6 + 2.4) (FIG. 5A). Serum ASM activity increased within a week of hASM-AAV injection and the higher level of ASM in the serum and muscle of the B6A/J mice treated with hASM-AAV was sustained even 12-weeks after treatment (FIGS. 5B, 5J, 7). This increased hASM had no adverse effect on the overall health of the animal or on the liver, as assessed by the growth of animals over the 12 weeks of treatment, by liver histopathology, and serum levels of Alanine Aminotransferase (ALT) (FIGS. 5B, 5C, 7). Given the demonstrable improvement imparted by hASM in muscle cell repair in vitro, it was assessed whether in vivo hASM increase improves sarcolemmal repair and reduced myofiber damage and degeneration in dysferlin-deficient muscle fibers. Using the presence of IgM within myofiber as an indicator of myofiber damage, hASM-AAV treatment led to a 3-fold reduction in the extent of damaged myofibers (FIGS. 5D, 5E). To directly assess the benefit of the in vivo hASM treatment on myofiber repair, an ex vivo laser injury assay was used for monitoring myofiber repair in intact biceps muscles from mice treated with control and hASM-AAV. Using this approach, improved repair ability of myofibers was observed from hASM-AAV treated mice as compared to those from control-AAV-treated mice (FIGS. 5E, 5F, 5G). As laser injury leads to a controlled and focal injury, the repair ability of myofibers injured via mechanical activity was examined. Repair from such mechanical injuries was assessed in intact EDL muscles from B6A/J mice treated with hASM-AAV or control AAV as well as age matched WT mice. The muscles were injured by 10 bouts of 10% eccentric contractions, followed by labeling the injured myofibers using the membrane impermeant vital dye, procion orange (PO). The mechanical injuries resulted in PO labeling of the injured myofibers that failed to repair, which was reduced by over 2-fold in hASM-AAV treated mice (FIGS. 5E, 5H). This improved myofiber repair of hASM-AAV treated mice correlated with the attenuation mechanical injury-induced muscle force loss, reaching the same level as the healthy (WT) muscle (FIG. 5I). These findings indicate that chronic in vivo exposure of dysferlinopathic muscle to elevated hASM restores myofiber repair capacity following spontaneous injuries in vivo, as well as focal, or mechanical injury ex vivo.

### Preclinical Benefits of hASM-AAV for LGMD2B

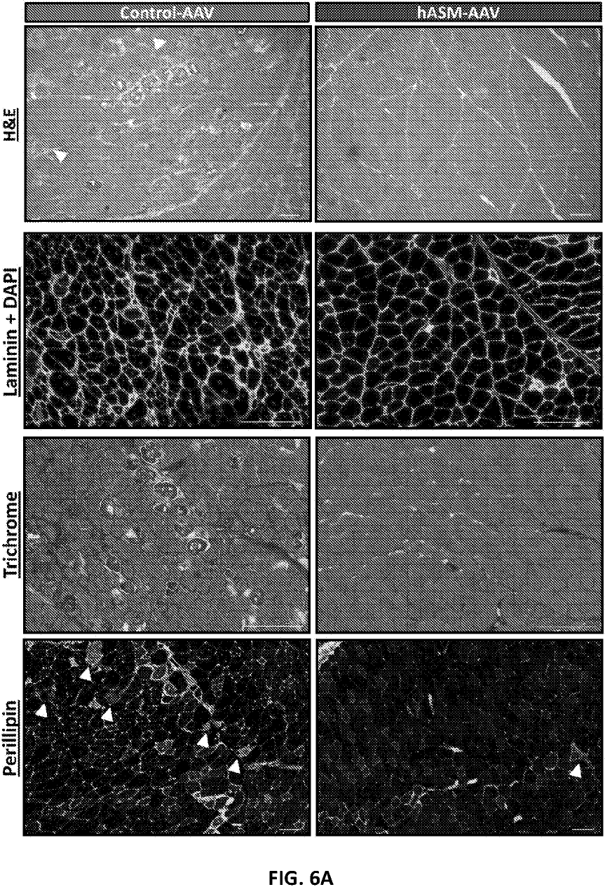

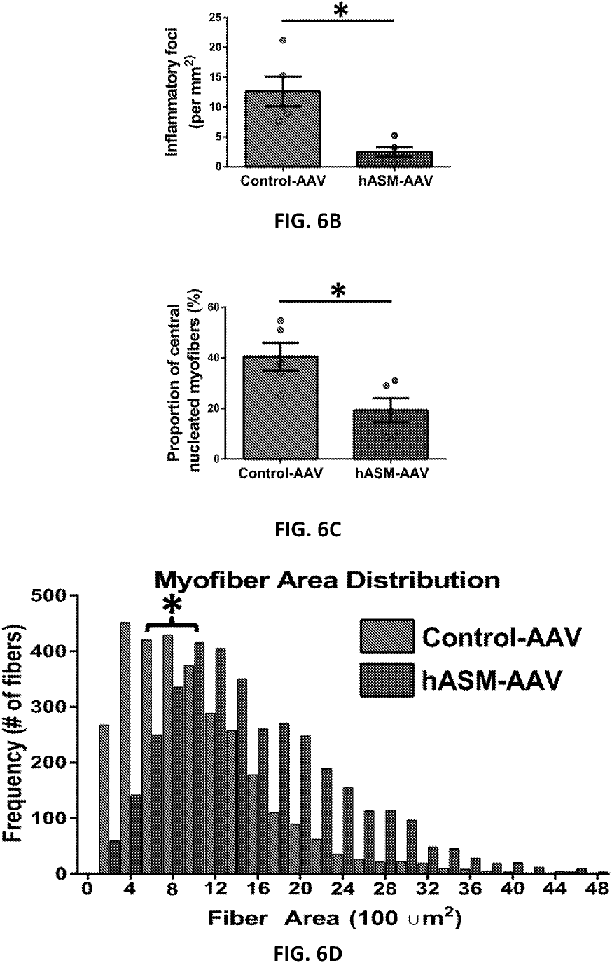

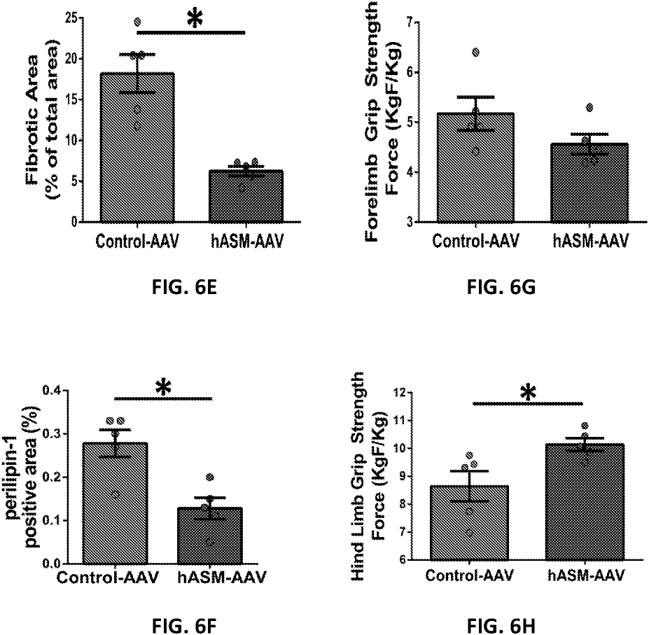

With the above beneficial effects of hASM-AAV therapy for treating the poor myofiber repair and excessive myofiber necrosis caused by dysferlin deficiency, it was then examined whether this treatment can also improve in vivo muscle histopathology and function. Quadriceps are the primary locomotor muscle group often affected by dysferlin deficiency (Ho, et al. (2004) Hum. Mol. Genet., 13(18): 1999-2010). Thus, the histopathology of these muscles was examined, which showed ~80% reduction in muscle inflammation (FIGS. 6A, 6B). Given that chronic skeletal muscle inflammation is damaging and leads to the need for greater regeneration, it was examined if reduced inflammation in hASM-AAV treated muscles also lowered regeneration. Quantification of the proportion of myofibers with central nuclei (a marker for regeneration) in the H&E labeled muscle section showed that hASM-AAV treatment reduced the need for myofiber regeneration by 2-fold (Control-AAV: 40.5% + 5.5, rhASM-AAV: 19.3% + 4.7) (FIG. 6C), which was confirmed by automated counting of centrally nucleated myofibers (FIG. 6A). Further, quantifying the cross-sectional area of ~3000 myofibers showed myofibers were ~45% larger in hASM-AAV-treatment condition as compared to the control-AAV (FIGS. 6A, 6D). Further, hASM-AAV treated muscles also showed a nearly 3-fold reduction in muscle fibrosis (Masson Trichrome staining) (FIGS. 6A, 6E), and adipogenic loss of the myofibers (Perilipin-1 staining) (FIGS. 6A, 6F). Finally, it was examined if these histopathological improvements by hASM-AAV treatment, led to any improvement in muscle strength. Dysferlin deficit causes greater force loss in the hindlimb muscles (Defour, et al. (2017) Human Mol. Genetics 26(11):1979-91), and improved membrane repair addresses this deficit (Sreetama et al. (2018) Molecular Therapy 26(9):2231-42). Thus, the forelimb and hindlimb muscle grip strength of the control and hASM-AAV treated mice were measured (Sreetama et al. (2018) Molecular Therapy 26(9):2231-42). It was found that compared to control-AAV treated cohort, while fore-limb grip strength was not appreciably altered, the hind-limb grip strength was significantly improved in the hASM-AAV treated cohort (FIGS. 6G, 6H). In light of these findings, it is apparent that single dose of hASM-AAV treatment allows extended histopathological and functional muscle improvement for LGMD2B.

Restoration of the cellular deficits downstream of the lack of dysferlin is a therapeutic approach for LGMD2B. To complement the ongoing gene therapy efforts aimed at restoring the expression of the large dysferlin protein in LGMD2B patient muscle, the work here provides an alternative approach. Using liver-targeted expression of a protein (ASM) nearly four-times smaller than dysferlin, the downstream consequence of dysferlin deficit is addressed. This offered preclinical benefits comparable to skeletal muscle dysferlin restoration. Dysferlin enables rapid and efficient lysosomal exocytosis required for timely secretion of ASM to help the injured muscle cells to repair frequent membrane injuries (Defour, et al. (2014) Cell Death Dis., 5:e1306). Insufficient ASM release by injured cells is a deficit common to both LGMD2B and NPDA patients (Defour, et al. (2014) Cell Death Dis., 5:e1306; Michailowsky, et al. (2019) Skelet. Muscle. 9(1):1). However, it was found that unlike NPDA, dysferlin deficient muscles do not lack ASM expression (FIG. 5J). Surprisingly, increased extracellular hASM improves muscle health in LGMD2B mouse model by, without being bound by theory, improving plasma membrane repair through enhanced CLIC-mediated endocytosis (FIGS. 2, 3). As CLIC facilitates dysferlin endocytosis (Hemandez-Deviez, et al. (2008) J. Biol. Chem., 283(10):6476-88) and localizes with pore forming toxins bound to the plasma membrane (Idone, et al. (2008) J. Cell. Biol., 180(5):905-14), CLIC endocytosis is intricately linked with plasma membrane repair, and as demonstrated here - ASM-mediated repair (Corrotte, et al. (2013) Elife 2:e00926).

Exogenous administration of hASM is safe for human use and shows therapeutic efficacy in treating symptoms caused by ASM deficit in NPD patients (Murray, et al. (2015) Mol. Genet. Metab., 114(2):217-25; Defour, et al. (2017) Human Mol. Genetics 26(11):1979-91). However, such studies have not assessed the capacity of hASM to improve membrane repair or evaluate its efficacy in treating LGMD2B — a disease caused not by the lack of ASM production, but by its reduced secretion. The studies here have examined the reparative properties of hASM and unexpectedly identified the efficacious extracellular dose of hASM that can restore membrane repair capacity in dysferlin deficient muscle cells (FIG. 1). This dose is lower than the dose that was used to enhance repair of ASM-deficient cells injured by pore-forming toxins (Tam, et al. (2010) J. Cell. Biol., 189(6): 1027-38). This hASM dose that is efficacious at improving plasma membrane repair is encouraging for its clinical utility in LGMD2B, as it is well below the established safe maximal hASM dose for use in humans and 100-fold lower than dose that induce cell death (FIG. 7) (McGovern, et al. (2016) Genet. Med., 18(1):34-40). However, given the short circulating half-life of injected hASM protein (21-24 hours), therapy relying on direct hASM delivery will require frequent administration and dose-escalation of the drug to maintain efficacy, thereby decreasing its utility to treat a chronic disease such as LGMD2B. To overcome this limitation, a more clinically feasible approach was tested of genetic delivery of hASM via hepatic expression of this protein by AAV-mediated delivery. With an excellent safety profile, and high transduction efficiency of AAVs in a broad range of tissues, there are over 2000 clinical trials to-date that utilize these gene transfer vectors (Kumar, et al. (2016) Mol. Ther. Methods Clin. Dev., 3:16034). Of these, liver-specific targeted AAV-based therapeutics offer greater efficacy of targeting by intravenous administration, allow multi-year transgene expression after single administration, and are efficient at treating plasma protein deficiencies (Nathwani, et al. (2014) N. Engl. J. Med., 371(21):1994-2004; Dobrzynski, et al. (2004) Blood 104(4):969-77; Cao, et al. (2007) Blood 110(4):1132-40; Colella, et al. (2018) Mol. Ther. Methods Clin. Dev., 8:87-104). Despite the safety of this approach, recent phase 1, 2 clinical trial (NCT03199469) for x-linked myotubular myopathy (MTM) utilizing AAV8 dose-escalation of MTM1 protein (AT132) observed detrimental hepatic outcome when high levels of viral load (1x10¹⁴ vg/kg up to 3x10¹⁴ vg/kg) was used. The study here shows therapeutic efficacy of hASM-AAV in mice at 1.1 x 10¹³ vg/kg (human equivalent dose of 1.1 x 10¹² vg/kg). This significantly lower human equivalent dose would enable safe use. Supporting this safety, no overt liver damage (serum ALT level and liver histopathology) was observed during and even at the end of the 12-week treatment with hASM-AAV.

Use of hASM-AAV in vitro showed that it allows production of secreted hASM by human liver cells (HepG2 cells) at levels that reached therapeutically efficacious concentrations and restores repair in dysferlin deficient patient muscle cells (FIG. 4). This efficacy is also reflected by the in vivo use of this vector in a preclinical mouse model of dysferlin deficiency. Use of a vector in the mouse model of NPD has demonstrated increased and stable hASM production in a 12-week study (Barbon, et al. (2005) Mol. Ther., 12(3):431-40). The in vivo use of hASM-AAV was shown using the LGMD2B mouse model where after 12 weeks of treatment with a single dose of this vector, there was detectable high levels of hepatic and serum hASM that was efficacious in restoring myofiber repair capacity (FIG. 5). These findings, while of interest for LGMD2B, is also of interest to NPDA patients since myofibers from mouse models for NPDA also manifest poor sacrolemmal repair (Michailowsky, et al. (2019) Skelet. Muscle 9(1):1).

Increased muscle degeneration necessitates greater muscle regeneration, and it was found that improved repair of dysferlin-deficient myofibers by hASM-AAV reduces the need for regeneration, causing a 2-fold decrease in the number of regenerated myofibers. It also decreased the proportion of small (newly regenerated) myofibers in the hASM-AAV treated mice (FIG. 6). These in vivo improvements by hASM-AAV are comparable to what is obtained by a Dysferlin-AAV mediated gene therapy approach (Potter, et al. (2018) Hum. Gene Ther., 29(7):749-62), and demonstrate that a secreted hASM-based gene therapy is as effective in rescuing the LGMD2B myofiber repair deficit as gene therapy that restored myofiber dysferlin expression. Additional consequences of persistent myofiber damage include chronic muscle inflammation (Tidball, et al. (2011) Compr. Physiol., 1(4):2029-62.). hASM-AAV treatment of dysferlin-deficient mice also attenuated this, arguably through the improved in vivo repair ability of the dysferlin-deficient myofibers (FIG. 6).

Continuous bouts of injury and poor membrane repair also promotes fibroadipogenic replacement of muscle. hASM-AAV caused reduced fibroadipogenic replacement of the dysferlinopathic muscle to an extent comparable to the reduction achieved using AAV-dysferlin gene therapy (Potter, et al. (2018) Hum. Gene Ther., 29(7):749-62) (FIG. 6). These findings indicated that the hASM-AAV treatment, in its capacity to improve muscle repair and overall muscle quality and health, would concomitantly preserve muscle function. In line with these improvements, increased hind-limb grip strength was observed following hASM-AAV treatment. Forelimb grip strength (which is unaffected in the LGMD2B mouse model (Lloyd, et al. (2019) PLoS One 14(4):e0214908)) was unaltered by the hASM-AAV treatment. Further, the observed improvement in hindlimb grip strength is in line with other pre-clinical approaches that offer therapeutic benefits for dysferlinopathy (Farini, et al. (2012) Exp. Cell Res., 318(10): 1160-74; Han, et al. (2010) J. Clin. Invest., 120(12):4366-74; Halevy, et al. (2013) Histol. Histopathol., 28(2):211-26.).

In summary, the results reported here demonstrate that hASM protein improves LGMD2B muscle cell sarcolemmal repair in a dose-dependent manner. They establish both purified hASM protein and AAV-mediated hepatic hASM gene transfer approaches as viable strategies for improving repair capacity of dysferlinopathic myofibers. Use of the gene transfer approach establishes its utility for longer-term in vivo benefits for reducing myofiber death and histopathology, as well as improving muscle function.

Lipid imbalance at the cellular and tissue levels characterize muscle degeneration in dysferlinopathy. Aberrant accumulation and adipogenic differentiation of fibroadipogenic cells causes muscle loss in dysferlinopathy. Targeting fibroadipogenic cells provides a therapeutic approach to curb muscle loss due to adipogenic degeneration. Genetically increasing secreted Acid Sphingomyelinase preserves dysferlinopathic muscle and prevents its functional decline.

A number of publications and patent documents are cited throughout the foregoing specification in order to describe the state of the art to which this invention pertains. The entire disclosure of each of these citations is incorporated by reference herein.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method of treating, inhibiting, and/or preventing a muscular dystrophy in a subject, said method comprising administering a nucleic acid encoding acid sphingomyelinase to said subject.

2. The method of claim 1, wherein said muscular dystrophy is a dysferlinopathy or is dysferlin deficient.

3. The method of claim 2, wherein said dysferlinopathy is limb-girdle muscular dystrophy type 2B (LGMD2B) or Miyoshi muscular dystrophy 1.

4. The method of claim 1, wherein said nucleic acid encoding acid sphingomyelinase is linked to a liver-specific or hepatocyte specific promoter.

5. The method of claim 4, wherein said liver-specific or hepatocyte specific protomer is selected from the group consisting of human a-1 antitrypsin (hAAT) promoter, hybrid liver promoter (HLP), human thyroxine-binding globulin (TBG), human serum albumin promoter, and DC190 promoter.

6. The method of claim 4, wherein said liver-specific or hepatocyte specific protomer is the human serum albumin promoter.

7. The method of claim 4, wherein said liver-specific or hepatocyte specific protomer is the DC190 promoter.

8. The method of claim 1, wherein said nucleic acid encoding acid sphingomyelinase is injected directly into the liver or into the bloodstream.

9. The method of claim 1, wherein said acid sphingomyelinase is human acid sphingomyelinase.

10. The method of claim 1, wherein said nucleic acid encoding acid sphingomyelinase is contained within a viral vector.

11. The method of claim 10, wherein said viral vector is an adeno-associated virus vector.
